(19) United States
(12) Patent Application Publication
(10) Pub. No.: US 2014/0154227 A1
(43) Pub. Date: Jun. 5, 2014

(54) CELL POPULATIONS HAVING IMMUNOREGULATORY ACTIVITY, METHOD FOR ISOLATION AND USES

(71) Applicants: Javier Garcia Casado, Ca'ceres (ES); Raquel Tarazona Lafarge, Ca'ceres (ES); Olga De La Rosa, Tres-Cantos (Madrid) (ES)

(72) Inventors: Javier Garcia Casado, Ca'ceres (ES); Raquel Tarazona Lafarge, Ca'ceres (ES); Olga De La Rosa, Tres-Cantos (Madrid) (ES)

(21) Appl. No.: 14/004,004

(22) PCT Filed: Mar. 12, 2012
(86) PCT No.: PCT/EP12/54251

§ 371 Date: Nov. 18, 2013

(30) Foreign Application Priority Data
Mar. 11, 2011 (EP) 11157930.6

(51) Int. Cl. C12N 5/0775

(52) U.S. Cl. CPC C12N 5/0667
USPC 424/93.71; 435/325; 435/7.21; 424/93.7; 435/375

(57) ABSTRACT

The present invention provides a population of mesenchymal cells that do not express the cell surface markers CD112 and/or CD155 for use in preventing, treating or ameliorating one or more symptoms associated with disorders in which modulation of a subject's immune system is beneficial, including, but not limited to, autoimmune diseases, inflammatory disorders, and immunologically mediated diseases including rejection of transplanted organs and tissues.

### FIELD OF THE INVENTION

The present invention relates to the prevention, treatment or amelioration of one or more symptoms of disorders in which modulation of a subject's immune system is beneficial utilizing cell populations derived from adult tissues. In particular, the present invention provides a population of mesenchymal stem cells that do not express the cell surface markers CD112 and/or CD155 for use in preventing, treating or ameliorating one or more symptoms associated with disorders in which modulation of a subject's immune system is beneficial, including, but not limited to, autoimmune diseases, inflammatory disorders, and immunologically mediated diseases including rejection of transplanted organs and tissues.

### BACKGROUND OF THE INVENTION

Mesenchymal stem cells (MSCs) are multipotent adult stem cells capable of differentiating into a variety of cell types. MSCs have been traditionally isolated from bone marrow but recent reports have performed the isolation and in vitro expansion from a variety of tissues including fetal liver and lung, adipose tissue, skeletal muscle], amniotic fluid, synovium, dental pulp, and skin. MSCs are thought to have tissue regenerative properties, in the first place, via their multilineage differentiation capacity and, more importantly, via the secretion of trophic factors that may activate local progenitor cells. MSCs also have potent immunomodulatory capacities, inhibiting the proliferation and cytotoxic potential of natural killer (NK) cells, T lymphocytes, γδT cells and invariant NKT cells. Moreover, MSCs have a limited efficiency of antigen processing and presentation and influence host immunity by modulating dendritic cell function.

Mesenchymal stem cells (hereinafter also referred to as hASCs) may be obtained from liposuction procedures and yield a clinically useful number of cells with characteristics of stem cells. These cells can be expanded over a long time in culture for clinical practice, being an interesting tool for cellular therapy. Therapeutic applications of hASCs are being explored and several clinical trials are on-going in graft-versus-host disease, fistula, Crohn's disease and urinary incontinence. The preclinical research activity of hASCs is currently being focused on diseases as diverse as diabetes, spinal cord injury, Huntington's disease, multiple sclerosis, ischemia, rheumatoid arthritis, skin regeneration, glioblastoma and colitis.

Although hASCs and Bone Marrow-Mesenchymal stem cells (hereafter referred to as hBM-MSCs) come from different sources they share functional similarities in their differentiation potential and their immunosuppressive mechanisms.

Despite the low immunogenicity of MSCs in vitro, one still has to be cautious in using MSCs in an allogeneic setting without immunosuppression of the patient. Given the significant role of MSCs over the adaptive immune system, for therapeutic applications it is interesting to clarify whether the immune privilege of the stem cells is maintained in the context of the innate response, especially in an allogeneic setting. In this sense, it has been reported by several groups that hBM-MSCs and dental pulp derived MSC (DP-MSC) can be lysed by cytotoxic immune effectors such as NK cells. The NK susceptibility of these cells may be due to the expression of ligands for activating receptors involving multiple interactions between NK and target cells. The recognition and lysis of allogeneic MSCs by NK cells have implications in safety (side effect associated with immune rejection) and efficacy (reduced persistence of the cells in the patient), for this, understanding the interaction of MSCs with NK cells is crucial to optimize their potential therapeutic use.

NK cells are a subset of lymphoid cells which have the capability of killing target cells without prior sensitization. The NK cell activation is mediated through specific interactions between activating receptors and their respective ligands. These activating receptors, once engaged, induce the lysis and cytokine release. On the contrary, to shift the balance towards NK cell inhibition, the activation of NK cells is prevented by inhibitory NK cell receptors.

Ligands for activating receptors such as DNAM-1 have been identified on the surface of hBM-MSCs cells and it has been demonstrated that activated NK cells are capable of killing hBM-MSCs and that NK receptor activation is involved.

The present invention provides an isolated population of mesenchymal stem cells that do not express NK receptor ligands for DNAM-1.

### SUMMARY OF THE INVENTION

The present invention provides cell populations with multilineage potential which are present in adipose tissues that are capable of acting as immunoregulatory agents. The inventors have isolated a population of mesenchymal stem cells that do not express the cell surface markers CD112 and/or CD155. The immunoregulatory effects of said cells can be used for preventing, treating or ameliorating one or more symptoms associated with disorders in which modulation of a subject's immune system is beneficial, including, but not limited to, autoimmune diseases, inflammatory disorders, and immunologically mediated diseases including rejection of transplanted organs and tissues. Although the use of mesenchymal stem cells as therapeutic agents is known in the art the herein disclosed cell population presents significant advantages over the hitherto disclosed cell populations in that it is not recognised by patient NK cells and therefore persists longer in the patient thereby potentially exerting a greater therapeutic effect.

Thus, in an aspect, the invention relates to mesenchymal stem cell population wherein the cells of said cell population do not express do not express the cell surface markers CD112 and/or CD155.

In another aspect, the invention relates to a method for the isolation of said cell population. The cell population obtainable according to said method constitutes an additional aspect of this invention.

In another aspect, the invention relates to said cell population for use in the prevention, treatment or amelioration of one or more symptoms of disorders in which modulation of a subject's immune system is beneficial.

In another aspect, the invention relates to said cell population for use as medicament, or for inducing transplantation tolerance, or for treating autoimmune diseases, or for treating an inflammatory disease, or for treating an immune-mediated inflammatory disease. In a particular embodiment, said inflammatory disease is a chronic inflammatory disease, such as, for example, Inflammatory Bowel Disease (IBD) or Rheumatoid Arthritis (RA).

In another aspect, the invention relates to the use of said cell population in the preparation of a medicament, such as a medicament for the prevention, treatment or amelioration of one or more symptoms of disorders in which modulation of a subject's immune system is beneficial, e.g., a medicament for inducing transplantation tolerance, or a medicament for treating autoimmune diseases, or a medicament for treating an inflammatory disease, or a medicament for treating an immune-mediated inflammatory disease.

In another aspect, the invention relates to the use of said cell population in the preparation or generation of regulatory T-cells (T-reg). Said T-reg cell population as well as a method for the isolation thereof constitute further aspects of the invention.

In another aspect, the invention relates to said T-reg cell population for use as medicament, or for inducing transplantation tolerance, or for treating autoimmune diseases, or for treating an inflammatory disease, or for treating an immune-mediated inflammatory disease.

In another aspect, the invention relates to the use of said T-reg cell population in the preparation of a medicament, such as a medicament for the prevention, treatment or amelioration of one or more symptoms of disorders in which modulation of a subject's immune system is beneficial, e.g., a medicament for inducing transplantation tolerance, or a medicament for treating autoimmune diseases, or a medicament for treating an inflammatory disease, or a medicament for treating an immune-mediated inflammatory disease or a medicament for treating allergies, for example, but not limited to, hypersensitivity Type IV reactions.

In another aspect, the invention relates to a method for the isolation of an irradiated cell population which comprises irradiating said cell population with a controlled source of ionizing radiation under appropriate conditions. Said irradiated cell population constitutes a further aspect of the invention.

In another aspect, the invention relates to said irradiated cell population for use as medicament, or for inducing transplantation tolerance, or for treating autoimmune diseases, or for treating an inflammatory disease, or for treating an immune-mediated inflammatory disease.

In another aspect, the invention relates to the use of said irradiated cell population in the preparation of a medicament, such as a medicament for the prevention, treatment or amelioration of one or more symptoms of disorders in which modulation of a subject's immune system is beneficial, e.g., a medicament for inducing transplantation tolerance, or a medicament for treating autoimmune diseases, or a medicament for treating an inflammatory disease, or a medicament for treating an immune-mediated inflammatory disease.

In another aspect, the invention relates to a method which comprises subjecting said cell population to treatment with interferon-γ (IFN-γ). Said IFN-γ-treated cell population constitutes a further aspect of the invention.

In another aspect, the invention relates to said IFN-γ-treated cell population for use as medicament, or for inducing transplantation tolerance, or for treating autoimmune diseases, or for treating an inflammatory disease, or for treating an immune-mediated inflammatory disease.

In another aspect, the invention relates to the use of said IFN-γ-treated cell population in the preparation of a medicament, such as a medicament for the prevention, treatment or amelioration of one or more symptoms of disorders in which modulation of a subject's immune system is beneficial, e.g., a medicament for inducing transplantation tolerance, or a medicament for treating autoimmune diseases, or a medicament for treating an inflammatory disease, or a medicament for treating an immune-mediated inflammatory disease.

In another aspect, the invention relates to a method which comprises subjecting said cell population to (i) irradiation, and (ii) stimulation with IFN-γ, wherein treatments (i) and (ii) are carried out in any order. Said irradiated IFN-γ-pre-stimulated cell population or IFN-γ-pre-stimulated irradiated cell population constitute a further aspect of the invention.

In another aspect, the invention relates to said irradiated IFN-γ-pre-stimulated cell population or IFN-γ-pre-stimulated irradiated cell population for use as medicament, or for inducing transplantation tolerance, or for treating autoimmune diseases, or for treating an inflammatory disease.

In another aspect, the invention relates to the use of said irradiated IFN-γ-pre-stimulated cell population or IFN-γ-pre-stimulated irradiated cell population in the preparation of a medicament, such as a medicament for the prevention, treatment or amelioration of one or more symptoms of disorders in which modulation of a subject's immune system is beneficial, e.g., a medicament for inducing transplantation tolerance, or a medicament for treating autoimmune diseases, or a medicament for treating an inflammatory disease, or a medicament for treating an immune-mediated inflammatory disease.

In another aspect, the invention relates to the use of said cell population, or said T-reg cell population, or said irradiated cell population, or said IFN-γ-treated cell population, or said irradiated IFN-γ-pre-stimulated cell population, or said IFN-γ-pre-stimulated irradiated cell population for preventing, treating, or ameliorating one or more symptoms associated with autoimmune diseases, inflammatory disorders, immune-mediated inflammatory disease or immunologically mediated diseases including rejection of transplanted organs and tissues.

In another aspect, the invention relates to a method of preventing, treating, or ameliorating one or more symptoms associated with autoimmune diseases, inflammatory disorders, or immunologically mediated diseases, in a subject suffering from any of said disorders or diseases, which comprises administering to said subject in need of such treatment of a prophylactically or therapeutically effective amount of said cell population, or said T-reg cell population, or said irradiated cell population, or said IFN-γ-treated cell population, or said irradiated IFN-γ-pre-stimulated cell population, or said IFN-γ-pre-stimulated irradiated cell population. The invention also relates to the use of such methods in combination therapy, in other words, a cell population of the invention is coadministered with one or more agents, either simultaneously with the second or further agent, or separately, e.g., sequentially.

In another aspect, the invention relates to a pharmaceutical composition comprising said cell population, or said T-reg cell population, or said irradiated cell population, or said IFN-γ-treated cell population, or said irradiated IFN-γ-pre-stimulated cell population, or said IFN-γ-pre-stimulated irradiated cell population and an acceptable pharmaceutically carrier.

In another aspect, the invention relates to a method for distinguishing adult multipotent cells from differentiated cells comprising the step of verifying whether the cell expresses the cell surface marker DNAM-1.

In another aspect, the invention relates to a kit comprising said cell population, or said T-reg cell population, or said irradiated cell population, or said IFN-γ-treated cell population, or said irradiated IFN-γ-pre-stimulated cell population, or said IFN-γ-pre-stimulated irradiated cell population.

### BRIEF DESCRIPTION OF THE FIGURES

FIG. 1. Expression of HLA molecules and ligands for NK activating receptors in hASCs and hBM-MSCs

hASCs and hBM-MSCs obtained from healthy donors were phenotypically characterized by multicolor flow cytometry. Quantification of HLA molecules and ligands for NK activating receptors is presented as Mean Relative Fluorescence Intensity (MRFI) calculated by dividing the Mean Fluorescent Intensity (MFI) by its negative control (numbers in brackets). The normalisation scores are determined from the fluorescence intensity according to the following keys: values <1.5=negative, >1.5<2=+/−, 2-10=+, >10<100=++, >100=+++. A representative histogram of each marker is represented both in the left column (hASCs) and right column (hBM-MSCs). Black bold histograms show the marker expression and empty lines represent the negative control Abbreviations: HLA-ABC, histocompatible locus antigen-ABC; HLA-DR, histocompatible locus antigen-DR; P, passage; ULBP, UL16-binding protein; NCR, natural cytotoxicity receptor; Fc, fragment crystallizable region of IgG; DNAM, DNAX accessory molecule-1.

FIG. 2. hASCs induce a low degranulation in IL-2-expanded NK cells.

Allogeneic PBMCs cells were pre-stimulated for 5 days with rhIL-2 and then sorted on the basis of CD56+CD3− phenotype. To quantify cytotoxic granule exocytosis, the surface expression of CD107a/b was analysed following activation of purified NK cells co-cultured with target cells (hASCs, hBM-MSCs, K562 or none) at 1:1 (NK:Target) ratio. The upper figure provides mean and standard deviation of the percentage of CD107a/b on CD56 positive cells. The lower row shows a representative dot plot of each condition. The numbers within the plots represent the percentage of CD107a/b on purified NK cells. Results are presented as a representative dot plot of five independent experiments. *p<0.05

FIG. 3. hASCs induce IFN-γproduction in IL-2-expanded NK cells.

Allogeneic PBMCs cells were pre-stimulated for 5 days with rhIL-2 and then sorted on the basis of CD56+CD3− phenotype. Following activation of purified NK cells with target cells (hASCs, hBM-MSCs, K562 or none) at 1:1 (NK:Target) ratio, intracellular staining was performed on NK cells using anti-human IFN-γ. Results are presented as mean±SD of six independent experiments together with a representative dot plot. A representative dot plot of each condition is represented in the figure and numbers in each plot indicate the percentage of IFN-γ on purified NK cells.

FIG. 4. The hASCs and hBM-MSCs decreased NK cell activity through contact-dependent and independent mechanisms.

Allogeneic NK cells sorted on the basis CD56+CD3− phenotype were co-cultured at ratio 1:1 in the presence or absence of hASCs or hBM-MSCs for 72 h in Transwell or direct contact. These cells were subsequently tested in a degranulation assay against a NK-susceptible target cell line (K562 cells). Upper graph represents degranulation of NK cells pre-incubated with hASCs. Lower graph represents degranulation of NK cells pre-incubated with hBM-MSCs. Values represent the mean±SD of 4 independently performed experiments. Abbreviations: NK_{hASCs}, natural killer presensitized with hASCs; NK_{hBM-MSCs}, natural killer presensitized with hBM-MSCs; NK_{control}, natural killer cultured alone. *p≦0.05

FIG. 5. NK cells promote the immunomodulatory activity of hASCs through IDO induction.

Upper and lower graph respectively shows tryptophan (Trp) and kynurenine (Kyn) concentration in supernatants from co-cultured NK/hASCs or NK/hBM-MSCs at ratio 1:1 for 72 h. Conditioned supernatants were measured by HPLC method. Black bars indicate mean and standard deviation of contact conditions and white bars indicates mean and standard deviation of transwell conditions. Values shown in the bars represent mean±SD of 4 independently performed experiments.

### DETAILED DESCRIPTION OF THE INVENTION

As has been previously mentioned, the inventors have isolated cell populations with multilineage potential which are present in mesenchymal derived tissues that do not express the cell surface markers CD112 and/or CD155 and are capable of acting as immunoregulatory agents. The immunosuppressant immunoregulatory effects of said cells can be used for preventing, treating or ameliorating one or more symptoms associated with disorders in which modulation of a subject's immune system is beneficial, including, but not limited to, autoimmune diseases, inflammatory disorders, and immunologically mediated diseases including rejection of transplanted organs and tissues.

### DEFINITIONS

In order to facilitate the understanding of the present description, the meaning of some terms and expressions in the context of the invention will be explained below. Further definitions will be included along the description when necessary.

As used herein the term “mesenchymal stem cell” (also referred to herein as “MSC”) shall be taken to mean a cell which is capable of giving rise to multiple different types of cell, originally derived from the mesenchyme. The term refers to a cell which is capable of differentiating into at least one of an osteoblast, a chondrocyte, an adipocyte, or a myocyte. MSCs may be isolated from any type of tissue. Generally MSCs will be isolated from bone marrow, adipose tissue, umbilical cord, or peripheral blood.

The term “immunoregulatory agent” refers to an agent that inhibits or reduces one or more biological activities of the immune system. An immunoregulatory agent is an agent that inhibits or reduces one or more biological activities (e.g., the proliferation, differentiation, priming, effector function, production of cytokines or expression of antigens) of one or more immune cells (e.g., T cells).

The term “immune disease” refers to a condition in a subject characterized by cellular, tissue and/or organ injury caused by an immunological reaction of the subject. The term “autoimmune disease” refers to a condition in a subject characterized by cellular, tissue and/or organ injury caused by an immunological reaction of the subject to its own cells, tissues and/or organs. Illustrative, non-limiting examples of autoimmune diseases which can be treated with the immunomodulatory cells of the invention include alopecia greata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, discoid lupus, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic thrombocytopenia purpura (ITP), IgA neuropathy, juvenile arthritis, lichen planus, Meniere's disease, mixed connective tissue disease, multiple sclerosis, type 1 or immune-mediated diabetes mellitus, myasthenia gravis, pemphigus vulgaris, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynauld's phenomenon, Reiter's syndrome, sarcoidosis, scleroderma, progressive systemic sclerosis, Sj ogren's syndrome, Good pasture's syndrome, stiff-man syndrome, systemic lupus erythematosus, lupus erythematosus, takayasu arteritis, temporal arteristis/giant cell arteritis, ulcerative colitis, uveitis, vasculitides such as dermatitis herpetiformis vasculitis, vitiligo, Wegener's granulomatosis, Anti-Glomerular Basement Membrane Disease, Antiphospholipid Syndrome, Autoimmune Diseases of the Nervous System, Familial Mediterranean Fever, Lambert-Eaton Myasthenic Syndrome, Sympathetic Ophthalmia, Polyendocrinopathies, Psoriasis, etc.

The term “Immune Mediated inflammatory Disease” shall be taken to mean any disease characterized by chronic or acute inflammation, resulting from, associated with or triggered by, a dysregulation of the normal immune response e.g. Crohn's disease, type 1 diabetes mellitus, rheumatoid arthritis, inflammatory bowel disease, psoriasis, psoriatic arthritis, ankylosing spondylitis, systemic lupus erythematosus, Hashimoto's disease, graft-versus-host disease, Sjogren's syndrome, pernicious anemia, Addison disease, scleroderma, Goodpasture's syndrome, ulcerative colitis, autoimmune hemolytic anemia, sterility, myasthenia gravis, multiple sclerosis, Basedow's disease, thrombopenia purpura, Guillain-Barré syndrome, allergy, asthma, atopic disease, arteriosclerosis, myocarditis, cardiomyopathy, glomerular nephritis, hypoplastic anemia, and rejection after organ transplantation.

“Celiac disease” is alternatively referred to as cceliac disease, c(o)eliac sprue, non-tropical sprue, endemic sprue, gluten enteropathy or gluten-sensitive enteropathy, and gluten intolerance.

For the purposes of the invention described herein, “immune disorders” include autoimmune diseases and immunologically mediated diseases.

The term “inflammatory disease” refers to a condition in a subject characterized by inflammation, e.g., chronic inflammation. Illustrative, non-limiting examples of inflammatory disorders include, but are not limited to, Celiac Disease, rheumatoid arthritis (RA), Inflammatory Bowel Disease (IBD), asthma, encephalitis, chronic obstructive pulmonary disease (COPD), inflammatory osteolysis, allergic disorders, septic shock, pulmonary fibrosis (e.g., idiopathic pulmonary fibrosis), inflammatory vacultides (e.g., polyarteritis nodosa, Wegner's granulomatosis, Takayasu's arteritis, temporal arteritis, and lymphomatoid granulomatosus), post-traumatic vascular angioplasty (e.g., restenosis after angioplasty), undifferentiated spondyloarthropathy, undifferentiated arthropathy, arthritis, inflammatory osteolysis, chronic hepatitis, and chronic inflammation resulting from chronic viral or bacteria infections.

The term “isolated” applied to a cell population refers to a cell population, isolated from the human or animal body, which is substantially free of one or more cell populations that are associated with said cell population in vivo or in vitro.

The term “MHC” (major histocompatibility complex) refers to a subset of genes that encodes cell-surface antigen-presenting proteins. In humans, these genes are referred to as human leukocyte antigen (HLA) genes. Herein, the abbreviations MHC or HLA are used interchangeably.

The term “subject” refers to an animal, preferably a mammal including a non-primate (e.g., a cow, pig, horse, cat, dog, rat, or mouse) and a primate (e.g., a monkey, or a human). In a preferred embodiment, the subject is a human.

The term “T-cell” refers to cells of the immune system which are a subset of lymphocytes that express the T cell receptor (TCR).

The term “regulatory T-cells” (T-reg cells) refers to T cell subsets that actively suppress activation of the immune system and prevent pathological self-reactivity, i.e. an autoimmune disease.

As used herein, the terms “treat”, “treatment” and “treating” refer to the amelioration of one or more symptoms associated with a disorder including, but not limited to, an inflammatory disorder, an autoimmune disease or an immunologically mediated disease including rejection of transplanted organs and tissues, that results from the administration of the cell population of the invention, the T-reg cell population of the invention, or the IFN-γ-pre-stimulated cell population of the invention, or a pharmaceutical composition comprising same, to a subject in need of said treatment.

The term “combination therapy” refers to the use of the cell populations of the present invention with other active agents or treatment modalities, in the manner of the present invention for the amelioration of one or more symptoms associated with a disorder including, but not limited to, an inflammatory disorder, an autoimmune disease or an immunologically mediated disease including rejection of transplanted organs and tissues. These other agents or treatments may include known drugs and therapies for the treatment of such disorders. The cell populations of the invention may also be combined with corticosteroids, non-steroidal anti-inflammatory compounds, or other agents useful in treating inflammation. The combined use of the agents of the present invention with these other therapies or treatment modalities may be concurrent, or given sequentially, that is, the two treatments may be divided up such that a cell population or a pharmaceutical composition comprising same of the present invention may be given prior to or after the other therapy or treatment modality. The attending physician may decide on the appropriate sequence of administering the cell population, or a pharmaceutical composition comprising same, in combination with other agents, therapy or treatment modality.

### Cells of the Invention

In one aspect, the present invention relates to an isolated mesenchymal stem cell population, hereinafter referred to as “cell population of the invention”, characterised in that the cells of said cell population do not express the markers CD112 and/or CD155. MSCs may be isolated from a number of mesenchyme derived and other tissues including but not limited to bone marrow, adipose tissue, umbilical cord, or peripheral blood. The MSCs used in the invention may in some embodiments preferably be isolated from bone marrow (BM-MSCs) or adipose tissue (ASCs). In a particularly preferred aspect of the invention, MSCs are obtained from lipoaspirates, themselves obtained from adipose tissue. The production of ASCs is known in the art, for example as described in WO-A-2006/136244.

In a preferred embodiment, the cells of the cell population of the invention are from a mammal, e.g., a rodent, primate, etc., preferably, from a human.

### Markers

The cells of the invention do not express and are therefore considered “negative” for the cell surface markers CD112 and/or CD155. Thus, the cells of the invention do not constitute a previously described subpopulation of mesenchymal stem cells.

Moreover, the cells of the invention are preferably negative for at least one, two of, or preferably all of the following cell surface markers: CD11b, CD11c, CD14, CD45, HLAII, CD31, CD34, CD45, 1B10 (αFSP), FceR1α and CD133.

As used herein the terms CD112 (IL2RB) and CD155 (PVR, Nectin 2) shall be taken to mean a polypeptide or fragment (including all splicing variants and isoforms) transcribed from the genomic sequence thereof (NM_{—}000878.2 (CD112) and NM_{—}001135768.1., NM_{—}001135769.1., NM_{—}001135770.1 or NM_{—}006505.3 (CD155)) and that has the capacity to bind to or function as a ligand of CD226 (DNAM-1).

In a preferred embodiment the term CD112 is taken to mean a polypeptide that comprises a sequence at least 85% identical (preferably, at least 90%, 95%, 98%, 99%, or 100% identical) to the amino acid sequence according to SEQ ID NO:1 or the extracellular region thereof (amino acids 27-240).

As used herein the term CD155 is taken to mean a polypeptide that comprises a sequence at least 85% identical (preferably, at least 90%, 95%, 98%, 99%, or 100% identical) to the amino acid sequence according to SEQ ID NO: 2 or the extracellular region thereof (amino acids 21-343).

As used herein, “negative” with respect to cell surface markers means that, in a cell population comprising the cells of the invention, less than 10%, preferably 9%, 8%, 7%, 6%, 5%, 4%, 3%, 2%, 1% or none of the cells show a signal for a specific cell surface marker in flow cytometry above the background signal, using conventional methods and apparatus (for example a Beckman Coulter Epics XL FACS system used with commercially available antibodies and standard protocols known in the art). In a particular embodiment, the cells of the invention are characterised in that they express at least one, two, three, four, of or preferably all of the following cell surface markers: CD9, CD44, CD54, CD90, CD29, CD59 and CD105; i.e., the cells of the invention are positive for at least one, two, three, four of and preferably all said cell surface markers (CD9, CD44, CD54, CD90, CD29, CD59 and CD105). Preferably, the cells of the invention are characterised in that they have significant expression levels of at least one, two, three, four, of and preferably all of said cell surface markers (CD9, CD44, CD54, CD90, CD29, CD59 and CD105). As used herein, the expression “significant expression” means that, in a cell population comprising the cells of the invention, more than 10%, preferably 20%, 30%, 40%, 50%, 60%, 70%, 80%, 90% or all of the cells show a signal for a specific cell surface marker in flow cytometry above the background signal using conventional methods and apparatus (for example .a Beckman Coulter Epics XL FACS system used with commercially available antibodies and standard protocols known in the art). The background signal is defined as the signal intensity given by a non-specific antibody of the same isotype as the specific antibody used to detect each surface marker in conventional FACS analysis. Thus for a marker to be considered positive the specific signal observed is stronger than 10%, preferably 20%, 30%, 40%, 50%, 60%, 70%, 80%, 90%, 500%, 1000%, 5000%, 10000% or above, than the background signal intensity using conventional methods and apparatus (for example .a Beckman Coulter Epics XL FACS system used with commercially available antibodies and standard protocols known in the art).

Optionally, the cells of the invention are also negative for the cell surface marker CD106 (VCAM-1).

Commercially available and known monoclonal antibodies against said cell-surface markers (e.g., cellular receptors and transmembrane proteins) can be used to identify the cells of the invention.

### Expression of IDO

The cells of the invention do not express IDO constitutively, but they express IDO upon stimulation with IFN-γ. Experiments carried out by the inventors have shown that said cells, upon stimulation with other pro-inflammatory mediators by themselves, such us interleukin-1 (IL-1) used at a concentration of 3 ng/ml, tumour necrosis factor-alpha (TNF-α) used at a concentration of 50 ng/ml, or the endotoxin LPS used at a concentration of 100 ng/ml, did not induce IDO expression, as measured by conventional RT-PCR and Western Blot analysis. Stimulation with IFN-γ for example at 3 ng/ml or higher can also induce expression of HLAII in the cells of the invention to give a positive signal as defined herein for a cell surface marker. Said expression can be detected by those skilled in the art using any known technique that allows the detection of the expression of specific proteins. Preferably, said techniques are cell cytometry techniques.

### Differentiation

The cells of the invention present the capacity to proliferate and be differentiated into at least two, more preferably three, four, five, six, seven or more cell lineages. Illustrative, non-limiting examples of cell lineages in which the cells of the invention can be differentiated include osteocytes, adipocytes, chondrocytes, tenocytes, myocytes, cardiomyocytes, hematopoietic-supporting stromal cells, endothelial cells, neurons, astrocytes, and hepatocytes.

Cells of the invention can proliferate and differentiate into cells of other lineages by conventional methods. Methods of identifying and subsequently isolating differentiated cells from their undifferentiated counterparts can be also carried out by methods well known in the art.

The cells of the invention are also capable of being expanded ex vivo. That is, after isolation, the cells of the invention can be maintained and allowed to proliferate ex vivo in culture medium. Such medium is composed of, for example, Dulbecco's Modified Eagle's Medium (DMEM), with antibiotics (for example, 100 units/ml Penicillin and 100 μg/ml Streptomycin) or without antibiotics, and 2 mM glutamine, and supplemented with 2-20% fetal bovine serum (FBS). It is within the skill of one in the art to modify or modulate concentrations of media and/or media supplements as necessary for the cells used. Sera often contain cellular and non-cellular factors and components that are necessary for viability and expansion. Examples of sera include FBS, bovine serum (BS), calf serum (CS), fetal calf serum (FCS), newborn calf serum (NCS), goat serum (GS), horse serum (HS), porcine serum, sheep serum, rabbit serum, rat serum (RS), etc. Also contemplated is, if the cells of the invention are of human origin, supplementation of cell culture medium with a human serum, preferably of autologous origin. It is understood that sera can be heat-inactivated at 55-65° C. if deemed necessary to inactivate components of the complement cascade. Modulation of serum concentrations, withdrawal of serum from the culture medium can also be used to promote survival of one or more desired cell types. Preferably, cells of the invention will benefit from FBS concentrations of about 2% to about 25%. In another embodiment, the cells of the invention can be expanded in a culture medium of definite composition, in which the serum is replaced by a combination of serum albumin, serum transferrin, selenium, and recombinant proteins including but not limited to: insulin, platelet-derived growth factor (PDGF), and basic fibroblast growth factor (bFGF) as known in the art.

Many cell culture media already contain amino acids; however some require supplementation prior to culturing cells. Such amino acids include, but are not limited to, L-alanine, L-arginine, L-aspartic acid, L-asparagine, L-cysteine, L-cystine, L-glutamic acid, L-glutamine, L-glycine, and the like.

Antimicrobial agents are also typically used in cell culture to mitigate bacterial, mycoplasmal, and fungal contamination. Typically, antibiotics or anti-mycotic compounds used are mixtures of penicillin/streptomycin, but can also include, but are not limited to amphotericin (Fungizone®), ampicillin, gentamicin, bleomycin, hygromacin, kanamycin, mitomycin, etc.

Hormones can also be advantageously used in cell culture and include, but are not limited to, D-aldosterone, diethylstilbestrol (DES), dexamethasone, b-estradiol, hydrocortisone, insulin, prolactin, progesterone, somatostatin/human growth hormone (HGH), etc.

The maintenance conditions of the cells of the invention can also contain cellular factors that allow cells to remain in an undifferentiated form. It is apparent to those skilled in the art that prior to differentiation, supplements that inhibit cell differentiation must be removed from the culture medium. It is also apparent that not all cells will require these factors. In fact, these factors may elicit unwanted effects, depending on the cell type.

Advantageously, the cells of the invention lack in vivo tumorigenic activity. Thus, said cells are characterized in that they do not present tumorigenic activity, i.e., they do not present an altered behaviour or proliferative phenotype which gives rise to a tumour cell

In an embodiment, the cells of the invention can be administered to a subject suffering from autoimmune diseases, inflammatory diseases or immunologically mediated diseases, such as rejection of transplanted organs and tissues, for suppressing the immune response. Thus, it is necessary that the cells of the invention do not present tumorigenic activity.

The tumorigenic activity of the cells of the invention can be tested by performing animal studies using immunodeficient mice strains. In these experiments, several million cells are implanted subcutaneously in the recipient animals, which are maintained for several weeks and analyzed for tumour formation. A particular assay is disclosed in Example 3.

The cells of the invention can be transfected or genetically engineered to express, at least, one antigenic polypeptide. In an embodiment, the antigen comprises a purified or a synthetic or recombinant polypeptide representing a specific antigen to which it is desired that tolerance is to be induced, or a short synthetic polypeptide fragment derived from the amino acid sequence of such an antigen. Preferably, the source of antigen comprises antigens expressed by a donor tissue graft. Also preferably, the source of antigen comprises a protein to which a patient has an autoimmune disorder.

### Method for Isolating Cells of the Invention

In an aspect, the present invention relates to a method for isolating a cell population from a tissue sample, wherein the cells of said cell population present a phenotype characterized in that (i) are negative for the markers CD 112 and/or CD 155; (ii) they present capacity to be differentiated into at least two cell lineages, said method comprising the steps of:
(i) preparing a cell suspension from a sample of tissue;
(ii) recovering the cells from said cell suspension;
(iii) incubating said cells in a suitable cell culture medium on a solid surface under conditions which allow cells to adhere to the solid surface and proliferate;
(iv) removing non-adhered cells;
(v) selecting the cells which after being passaged at least twice in such medium remain adhered to said solid surface; and
(vi) determining the presence or absence of determining the presence or absence of markers CD112 and/or CD155 in individual cells or subpopulations
(vii) selecting the cells or subpopulations negative for the markers CD112 and/or CD155.

As used herein, the term “solid surface” refers to any material that allows the cells of the invention to adhere. In a particular embodiment said material is a plastic material treated to promote the adhesion of mammalian cells to its surface, for example commercially available polystyrene plates optionally coated with poly-D-Lysine or other reagents.

Steps (i)-(vii) can be carried out by conventional techniques known by those skilled in the art. Briefly, the cells of the invention can be obtained by conventional means from any suitable source of connective tissue from any suitable animal, preferably humans, e.g., from human adipose tissue. The animal can be alive or dead, so long as connective tissue cells within the animal are viable. Typically, human adipose cells are obtained from living donors, using well-recognized protocols such as surgical or suction lipectomy. Indeed, as liposuction procedures are so common, liposuction effluent is a particularly preferred source from which the cells of the invention can be derived. Thus, in a particular embodiment, the cells of the invention are from the stromal fraction of human adipose tissue obtained by liposuction.

The sample of adipose tissue is, preferably, washed before being processed to separate the cells of the invention from the remainder of the material. In a protocol, the sample of tissue is washed with physiologically-compatible saline solution (e.g., phosphate buffered saline (PBS)) and then vigorously agitated and left to settle, a step that removes loose matter (e.g., damaged tissue, blood, erythrocytes, etc) from the tissue. Thus, the washing and settling steps generally are repeated until the supernatant is relatively clear of debris. The remaining cells generally will be present in clumps of various sizes, and the protocol proceeds using steps gauged to degrade the gross structure while minimizing damage to the cells themselves. One method of achieving this end is to treat the washed lumps of cells with an enzyme that weakens or destroys bonds between cells (e.g., collagenase, dispase, trypsin, etc.). The amount and duration of such enzymatic treatment will vary, depending on the conditions employed, but the use of such enzymes is generally known in the art. Alternatively or in conjunction with such enzymatic treatment, the lumps of cells can be degraded using other treatments, such as mechanical agitation, sonic energy, thermal energy, etc. If degradation is accomplished by enzymatic methods, it is desirable to neutralize the enzyme following a suitable period, to minimize deleterious effects on the cells.

The degradation step typically produces a slurry or suspension of aggregated cells and a fluid fraction containing generally free stromal cells (e.g., red blood cells, smooth muscle cells, endothelial cells, fibroblast cells, and stem cells). The next stage in the separation process is to separate the aggregated cells from the cells of the invention. This can be accomplished by centrifugation, which forces the cells into a pellet covered by a supernatant. The supernatant then can be discarded and the pellet suspended in a physiologically-compatible fluid. Moreover, the suspended cells typically include erythrocytes, and in most protocols it is desirable to lyse them. Methods for selectively lysing erythrocytes are known in the art, and any suitable protocol can be employed (e.g., incubation in a hyper- or hypotonic medium, by lysis using ammonium chloride, etc.). Of course, if the erythrocytes are lysed, the remaining cells should then be separated from the lysate, for example by filtration, sedimentation, or density fractionation.

Regardless of whether the erythrocytes are lysed, the suspended cells can be washed, re-centrifuged, and resuspended one or more successive times to achieve greater purity. Alternatively, the cells can be separated on the basis of cell surface marker profile or on the basis of cell size and granularity.

Following the final isolation and resuspension, the cells can be cultured and, if desired, assayed for number and viability to assess the yield. Preferably, the cells will be cultured without differentiation, on a solid surface, using a suitable cell culture media, at the appropriate cell densities and culture conditions. Thus, in a particular embodiment, cells are cultured without differentiation on a solid surface, usually made of a plastic material, such as Petri dishes or cell culture flasks, in the presence of a suitable cell culture medium [e.g., DMEM, typically supplemented with 5-15% (e.g., 10%) of a suitable serum, such as fetal bovine serum or human serum], and incubated under conditions which allow cells to adhere to the solid surface and proliferate. After incubation, cells are washed in order to remove non-adhered cells and cell fragments. The cells are maintained in culture in the same medium and under the same conditions until they reach the adequate confluence, typically, about 80% cell confluence, with replacement of the cell culture medium when necessary. After reaching the desired cell confluence, the cells can be expanded by means of consecutive passages using a detachment agent such as trypsin and seeding onto a bigger cell culture surface at the appropriate cell density (usually 2,000-10,000 cells/cm²). Thus, cells are then passaged at least two times in such medium without differentiating, while still retaining their developmental phenotype, and more preferably, the cells can be passaged at least 10 times (e.g., at least 15 times or even at least 20 times) without losing developmental phenotype. Typically, the cells are plated at a desired density such as between about 100 cells/cm² to about 100,000 cells/cm² (such as about 500 cells/cm² to about 50,000 cells/cm², or, more particularly, between about 1,000 cells/cm² to about 20,000 cells/cm²). If plated at lower densities (e.g., about 300 cells/cm²), the cells can be more easily clonally isolated. For example, after a few days, cells plated at such densities will proliferate into an homogeneous population. In a particular embodiment, the cell density is between 2,000-10,000 cells/cm².

Cells which remain adhered to the solid surface after such treatment comprising at least two passages are selected and the expression of the markers CD112 and/or CD155 is analyzed by conventional methods in order to confirm the identity of the cells of the invention. Cells which remain adhered to the solid surface after the first passage are from heterogeneous origin; therefore, said cells must be subjected to at least another passage. Cells which remain adhered to the solid surface after such treatment comprising at least two passages are selected and the expression of the markers CD112 and/or CD155 is analyzed by conventional methods in order to confirm the identity of the cells of the invention. As a result of the above method, a homogeneous cell population having the phenotype of interest is obtained.

Cell-surface markers can be identified by any suitable conventional technique, usually based on a positive/negative selection; for example, monoclonal antibodies against cell-surface markers, whose presence/absence in the cells has to be confirmed, can be used; although other techniques can also be used. Thus, in a particular embodiment, monoclonal antibodies against CD112 and/or CD155 are used in order to confirm the absence of said markers in the selected cells. In a further embodiment monoclonal antibodies against one, two, three, four, five, six, seven of or preferably all of CD11b, CD11c, CD14, CD45, HLAII, CD31, CD34, CD45, 1B10 (αFSP), FceR1α and CD133 are used in order to confirm the absence of said markers in the selected cells; and monoclonal antibodies against one, two, three, four, of or preferably all of CD9, CD44, CD54, CD90, CD29, CD59 and CD105 are used in order to confirm the presence thereof or detectable expression levels of, at least one of and preferably all of, said markers. Said monoclonal antibodies are known, commercially available or can be obtained by a skilled person in the art by conventional methods.

The capacity of the selected cells to differentiate into at least two cell lineages can be assayed by conventional methods as known in the art.

The cells and cell populations provided by the instant invention are preferably clonally expanded, using a suitable method for cloning cell populations. For example, a proliferated population of cells can be physically picked and seeded into a separate plate (or the well of a multi-well plate). Alternatively, the cells can be subcloned onto a multi-well plate at a statistical ratio for facilitating placing a single cell into each well (e.g., from about 0.1 to about 1 cell/well or even about 0.25 to about 0.5 cells/well, such as 0.5 cells/well). Of course, the cells can be cloned by plating them at low density (e.g., in a Petri dish or other suitable substrate) and isolating them from other cells using devices such as a cloning rings. The production of a clonal population can be expanded in any suitable culture medium. In any event, the isolated cells can be cultured to a suitable point when their developmental phenotype can be assessed.

It is known in the art that ex vivo expansion of the cells of the invention without inducing differentiation can be accomplished for extended time periods for example by using specially screened lots of suitable serum (such as fetal bovine serum or human serum). Methods for measuring viability and yield are known in the art (e.g., trypan blue exclusion).

Any of the steps and procedures for isolating the cells of the cell population of the invention can be performed manually, if desired. Alternatively, the process of isolating such cells can be facilitated and/or automated through one or more suitable devices, examples of which are known in the art.

### Irradiated Cells of the Invention

If desired, the cells of the invention can be irradiated using a suitable controlled source of ionizing radiation, such a gamma irradiator device. The irradiation conditions must be experimentally adjusted by a person skilled in the art to determine the required exposure time to impart a radiation dose that cause the long term growth arrest of the cells of the invention. Said radiation dose can be for example 1-100, 5-85, 10-70, 12-60 Gy or more preferably 15-45 Gy.

Since the cells of the invention can be used for therapeutic uses, irradiation of the cells of the invention before administration to the subject may result beneficial since said irradiation treatment makes cells incapable to proliferate or survive for long time periods in the subject. Said irradiated cells constitute a further aspect of the instant invention.

The irradiated cells of the invention can be used for preventing, treating or ameliorating one or more symptoms associated with disorders in which modulation of a subject's immune system is beneficial, including, but not limited to, autoimmune diseases, inflammatory disorders, and immunologically mediated diseases including rejection of transplanted organs and tissues. Said use constitutes an additional aspect of the present invention.

Thus, in another aspect, the irradiated cells of the invention are used as a medicament. In a particular embodiment, medicaments containing the irradiated cells of the invention may be used for inducing transplantation tolerance, or for treating, and thereby alleviating, symptoms of autoimmune or inflammatory disorders, or immunologically mediated diseases including rejection of transplanted organs and tissues, in a subject suffering from any of said disorders or diseases. Thus, the irradiated cells of the invention can be used to therapeutically or prophylactically treat and thereby alleviating symptoms of autoimmune or inflammatory disorders in a subject suffering from any of said disorders or to alleviate symptoms of immunologically mediated diseases in a subject suffering from said diseases.

Practically any autoimmune disease, inflammatory disorder or immunological mediated disease can be treated with the irradiated cells of the invention. Illustrative, non-limiting examples of said diseases and disorders which can be treated are those previously listed under heading “Definitions”. In a particular embodiment, said inflammatory disease is a chronic inflammatory disease, such as, e.g., IBD or RA.

In another aspect, the present invention relates to the use of the irradiated cells of the invention for the preparation of a medicament for preventing, treating or ameliorating one or more symptoms associated with disorders in which modulation of a subject's immune system is beneficial, including, but not limited to, autoimmune diseases, inflammatory disorders, and immunologically mediated diseases including rejection of transplanted organs and tissues. Thus, the invention further refers to the use of the irradiated cells of the invention for the preparation of a medicament for suppressing the immune response, or for inducing transplantation tolerance, or for treating autoimmune diseases, or for treating inflammatory disorders. Examples of said autoimmune diseases and inflammatory diseases have been previously mentioned. In a particular embodiment, disease is an inflammatory disease, such as a chronic inflammatory disease, e.g., IBD or RA.

### IFN-γ-Pre-Stimulated Cells of the Invention

Also, if desired, the cells of the invention can be pre-stimulated with IFN-γ. The methods for pre-stimulation with IFN-γ are evident to those skilled in the art. Preferably, the cells are pre-stimulated using a concentration of IFN-γ between 0.1 and 100, 0.5 and 85, 1 and 70, 1.5 and 50, 2.5 and 40 ng/ml or more preferably 3 and 30 ng/ml, and a stimulation time preferably longer than 12 hours, for example, 13, 18, 24, 48, 72 hours or more.

Since the cells of the invention can be used for therapeutic uses, pre-stimulation of the cells of the invention with IFN-γbefore administration to the subject may result beneficial since the time period between IFN-γ-pre-stimulated cell administration and IDO expression in the subject can be reduced.

Thus, in another aspect, the present invention refers to a method which comprises the treatment of the cells of the invention with IFN-γ in order to pre-stimulate said cells. The cells obtainable according to said method, hereinafter referred to “IFN-γ-pre-stimulated cells of the invention”, constitutes an additional aspect of the present invention. The IFN-γ-pre-stimulated cells of the invention can be isolated by conventional means known by a skilled person in the art.

The IFN-γ-pre-stimulated cells of the invention can be used for preventing, treating or ameliorating one or more symptoms associated with disorders in which modulation of a subject's immune system is beneficial, including, but not limited to, autoimmune diseases, inflammatory disorders, and immunologically mediated diseases including rejection of transplanted organs and tissues. Said use constitutes an additional aspect of the present invention.

Thus, in another aspect, the IFN-γ-pre-stimulated cells of the invention are used as a medicament. In a particular embodiment, medicaments containing the IFN-γ-pre-stimulated cells of the invention may be used for inducing transplantation tolerance, or for treating, and thereby alleviating, symptoms of autoimmune or inflammatory disorders, or immunologically mediated diseases including rejection of transplanted organs and tissues, in a subject suffering from any of said disorders or diseases. Thus, the IFN-γ-pre-stimulated cells of the invention can be used to therapeutically or prophylactically treat and thereby alleviating symptoms of autoimmune or inflammatory disorders in a subject suffering from any of said disorders or to alleviate symptoms of immunologically mediated diseases in a subject suffering from said diseases.

Practically any autoimmune disease, inflammatory disorder or immunological mediated disease can be treated with the IFN-γ-pre-stimulated cells of the invention. Illustrative, non-limiting examples of said diseases and disorders which can be treated are those previously listed under heading “Definitions”. In a particular embodiment, said inflammatory disease is a chronic inflammatory disease, such as, e.g., IBD or RA.

In another aspect, the present invention relates to the use of the IFN-γ-pre-stimulated cells of the invention for the preparation of a medicament for preventing, treating or ameliorating one or more symptoms associated with disorders in which modulation of a subject's immune system is beneficial, including, but not limited to, autoimmune diseases, inflammatory disorders, and immunologically mediated diseases including rejection of transplanted organs and tissues. Thus, the invention further refers to the use of the IFN-γ-pre-stimulated cells of the invention for the preparation of a medicament for suppressing the immune response, or for inducing transplantation tolerance, or for treating autoimmune diseases, or for treating inflammatory disorders. Examples of said autoimmune diseases and inflammatory diseases have been previously mentioned. In a particular embodiment, disease is an inflammatory disease, such as a chronic inflammatory disease, e.g., IBD or RA.

### Irradiated IFN-γ-Pre-Stimulated Cells of the Invention and IFN-γ-Pre-Stimulated Irradiated Cells of the Invention

Furthermore, if desired, the cells of the invention can be subjected to the treatments of irradiation and IFN-γ-stimulation, in any order; i.e., cells of the invention can be subjected firstly to irradiation and the resulting cells can be subsequently subjected to IFN-γ-stimulation, or vice versa, cells of the invention can be subjected firstly to IFN-γ-stimulation and subsequently the resulting cells can be subjected to irradiation.

Thus, in an aspect, the cells of the invention can be pre-stimulated with IFN-γ and the resulting cells (IFN-γ-pre-stimulated cells of the invention) can be irradiated to render irradiated cells hereinafter referred to as “irradiated IFN-γ-pre-stimulated cells of the invention”.

In another aspect, the cells of the invention can be irradiated and the resulting cells (irradiated cells of the invention) can be pre-stimulated with IFN-γ to render IFN-γ-prestimulated cells hereinafter referred to as “IFN-γ-pre-stimulated irradiated cells of the invention”.

Methods for pre-stimulation cells with IFN-γ as well as methods for irradiating cells are well-known for those skilled in the art and some of them have been previously mentioned above. Any of said methods can be used.

Thus, in another aspect, the present invention refers to a method which comprises subjecting the cells of the invention to (i) irradiation, and (ii) stimulation with IFN-γ, wherein treatments (i) and (ii) can be carried out in any order, in order to irradiate IFN-γ-pre-stimulated cells or to INF-γ-pre-stimulate irradiated cells. The cells obtainable according to said method, herein referred to as “irradiated IFN-γ-pre-stimulated cells of the invention” or “IFN-γ-pre-stimulated irradiated cells of the invention”, respectively, constitutes additional aspects of the present invention. Said irradiated IFN-γ-pre-stimulated cells of the invention as well as said IFN-γ-pre-stimulated irradiated cells of the invention can be isolated by conventional means known by a skilled person in the art.

Since the cells of the invention can be used for therapeutic uses, administration to a subject of the cells of the invention previously subjected to irradiation and IFN-γ-stimulation, in any order, may result beneficial for the reasons previously mentioned (e.g., subjecting cells to an irradiation treatment to make the cells incapable of proliferating or surviving for long time periods in the subject, whereas pre-stimulation of cells with IFN-γ before administration to the subject may involve a reduction in the time period between IFN-γ-pre-stimulated cell administration and IDO expression in the subject.

The irradiated IFN-γ-pre-stimulated cells of the invention as well as the IFN-γ-pre-stimulated irradiated cells of the invention can be used for preventing, treating or ameliorating one or more symptoms associated with disorders in which modulation of a subject's immune system is beneficial, including, but not limited to, autoimmune diseases, inflammatory disorders, and immunologically mediated diseases including rejection of transplanted organs and tissues. Said use constitutes an additional aspect of the present invention.

Thus, in another aspect, the irradiated IFN-γ-pre-stimulated cells of the invention as well as the IFN-γ-pre-stimulated irradiated cells of the invention are used as a medicament. In a particular embodiment, medicaments containing the irradiated IFN-γ-pre-stimulated cells of the invention or the IFN-γ-pre-stimulated irradiated cells of the invention may be used for inducing transplantation tolerance, or for treating, and thereby alleviating, symptoms of autoimmune or inflammatory disorders, or immunologically mediated diseases including rejection of transplanted organs and tissues, in a subject suffering from any of said disorders or diseases. Thus, the irradiated IFN-γ-pre-stimulated cells of the invention as well as the IFN-γ-pre-stimulated irradiated cells of the invention can be used to therapeutically or prophylactically treat and thereby alleviating symptoms of autoimmune or inflammatory disorders in a subject suffering from any of said disorders or to alleviate symptoms of immunologically mediated diseases in a subject suffering from said diseases.

Practically any autoimmune disease, inflammatory disorder or immunological mediated disease can be treated with the irradiated IFN-γ-pre-stimulated cells of the invention or with the IFN-γ-pre-stimulated irradiated cells of the invention. Illustrative, non-limiting examples of said diseases and disorders which can be treated are those previously listed under heading “Definitions”. In a particular embodiment, said inflammatory disease is a chronic inflammatory disease, such as, e.g., IBD or RA.

In another aspect, the present invention relates to the use of the irradiated IFN-γ-pre-stimulated cells of the invention or the IFN-γ-pre-stimulated irradiated cells of the invention for the preparation of a medicament for preventing, treating or ameliorating one or more symptoms associated with disorders in which modulation of a subject's immune system is beneficial, including, but not limited to, autoimmune diseases, inflammatory disorders, and immunologically mediated diseases including rejection of transplanted organs and tissues. Thus, the invention further refers to the use of the irradiated IFN-γ-pre-stimulated cells of the invention or the IFN-γ-pre-stimulated irradiated cells of the invention for the preparation of a medicament for suppressing the immune response, or for inducing transplantation tolerance, or for treating autoimmune diseases, or for treating inflammatory disorders. Examples of said autoimmune diseases and inflammatory diseases have been previously mentioned. In a particular embodiment, disease is an inflammatory disease, such as a chronic inflammatory disease, e.g., IBD or RA.

### T-Reg Cells of the Invention

The invention further refers, In another aspect, to regulatory T-cells (T-reg), i.e., cells (including Foxp3+CD4+CD25+T-reg and IL-10/TGFb-producing Trl cells) that actively suppress activation of the immune system and prevent pathological self-reactivity, i.e. an autoimmune disease, obtainable from the cells of the invention, hereinafter referred to T-reg cells of the invention.

Thus, In another aspect, the present invention relates to a method for the isolation of a T-reg cell population of the invention, which comprises:
(a) contacting a cell population of the invention with peripheral blood leukocytes, and
(b) selecting the T-reg cell population of the invention.

Consequently, the cells of the invention can be used to produce a subset of T-cells, the T-reg cells of the invention, which constitutes an additional aspect of the present invention. The T-reg cells of the invention can be isolated by conventional means known by a skilled person in the art.

The T-reg cells of the invention can be used for preventing, treating or ameliorating one or more symptoms associated with disorders in which modulation of a subject's immune system is beneficial, including, but not limited to, autoimmune diseases, inflammatory disorders, and immunologically mediated diseases including rejection of transplanted organs and tissues. Said use constitutes an additional aspect of the present invention.

Thus, in another aspect, the T-reg cells of the invention are used as a medicament. In a particular embodiment, medicaments containing the T-reg cells of the invention may be used for inducing transplantation tolerance, or for treating, and thereby alleviating, symptoms of autoimmune or inflammatory disorders, or immunologically mediated diseases including rejection of transplanted organs and tissues, in a subject suffering from any of said disorders or diseases. Thus, the T-reg cells of the invention can be used to therapeutically or prophylactically treat and thereby alleviating symptoms of autoimmune or inflammatory disorders in a subject suffering from any of said disorders or to alleviate symptoms of immunologically mediated diseases in a subject suffering from said diseases.

Practically any autoimmune disease, inflammatory disorder or immunological mediated disease can be treated with the T-reg cells of the invention. Illustrative, non-limiting examples of said diseases and disorders which can be treated are those previously listed under heading “Definitions”. In a particular embodiment, said inflammatory disease is a chronic inflammatory disease, such as, e.g., IBD or RA.

In another aspect, the present invention relates to the use of the T-reg cells of the invention for the preparation of a medicament for preventing, treating or ameliorating one or more symptoms associated with disorders in which modulation of a subject's immune system is beneficial, including, but not limited to, autoimmune diseases, inflammatory disorders, and immunologically mediated diseases including rejection of transplanted organs and tissues. Thus, the invention further refers to the use of the T-reg cells of the invention for the preparation of a medicament for suppressing the immune response, or for inducing transplantation tolerance, or for treating autoimmune diseases, or for treating inflammatory disorders. Examples of said autoimmune diseases and inflammatory diseases have been previously mentioned. In a particular embodiment, disease is an inflammatory disease, such as a chronic inflammatory disease, e.g., IBD or RA.

The invention also provides the use of cell populations of the invention in the production of Treg cells specific for a chosen antigen or group of antigens and the use of these in the treatment of disease or disorders relating to that antigen or group of antigens. Examples of such antigens are those that play a role in autoimmune diseases, such as, for example, rheumatoid arthritis, Crohn's disease, hypersensitivity reaction Type IV, lupus, psoriasis and other autoimmune disorders known in the art and described elsewhere herein. Briefly, cell populations of the invention are cultured in vitro in the presence of a chosen antigen, group of antigens or cell types expressing and/or presenting this antigen or antigens. The cells of the invention can optionally be prestimulated with IFNγ, LPS or other activating agents known in the art. After a culture period of about 2, 4, 6, 12, 24, 48 or more hours, preferably between about 12 to about 24 hours, the cell population of the invention is further co-cultured, optionally after the removal of the antigen, group of antigens or cells carrying said antigen, with peripheral blood leukocytes obtained from a subject. This co-culturing will result in the production of Treg cells specific for the chosen antigen, which can be used for treatment of the subject. Optionally these Treg cells can be expanded in number ex vivo using culture techniques known in the art before being administered to the patient. Without wishing to be bound by theory, the Inventors believe that the cell populations of the invention are capable of presenting the chosen antigen via HLA Class II on the cell surface (seeming induced by IFNγ) to the peripheral blood leukocytes such that Treg cells are augmented and or activated within the population of peripheral blood leukocytes. As shown in Example 11, the Inventors have demonstrated that cell populations of the invention are able to phagocytose small molecular weight molecules and thus are capable of presenting such molecules after IFNγ stimulation via HLA Class II molecules. The presentation of chosen antigen via this mechanism with the interaction with the peripheral blood leukocytes is believed to result in the above described Treg cell production. As an alternative treatment methodology, as described in Example 7 a cell population of invention is administered directly in vivo without any co-culturing and can generate specific Treg cells, which in turn can treat a disorder.

Thus the invention provides an in vitro method of obtaining Treg cells specific for a chosen antigen or group of antigens, which comprises:

(a) contacting a cell population of the invention with said chosen antigen or group of antigens;

(b) bringing said cell population into contact with peripheral blood leukocytes;

(c) selecting a T-reg cell population specific for said chosen antigen or group of antigens

The invention also provides the use of the specific Treg cells of step (c) in the treatment of diseases and disorders related to said chosen antigen or groups of antigens by administration of said Treg cells to the subject from which the peripheral blood leukocytes were obtained. The cell population of the invention as used in this method may be from the subject (autologous) or from a donor (allogeneic).

### Pharmaceutical Compositions

The present invention provides pharmaceutical compositions for the treatment, prophylaxis, and amelioration of one or more symptoms associated with a disorder in which modulation of a subject's immune system is beneficial such as autoimmune diseases, inflammatory disorders, and immunologically mediated diseases including rejection of transplanted organs and tissues.

Thus, in another aspect, the invention relates to a pharmaceutical composition, hereinafter referred to as the pharmaceutical composition of the invention, comprising a cell of the invention, or a T-reg cell of the invention, or an irradiated cell of the invention, or an IFN-γ-pre-stimulated cell of the invention, or an irradiated IFN-γ-pre-stimulated cell of the invention, or an IFN-γ-pre-stimulated irradiated cell of the invention, and an acceptable pharmaceutically carrier. Combinations of two or more of said type of cells are included within the scope of the pharmaceutical compositions provided by the instant invention.

The pharmaceutical composition of the invention comprises a prophylactically or therapeutically effective amount of one or more prophylactic or therapeutic agents (i.e., cell of the invention, or a T-reg cell of the invention, or an irradiated cell of the invention, or an IFN-γ-pre-stimulated cell of the invention, or an irradiated IFN-γ-pre-stimulated cell of the invention, or an IFN-γ-pre-stimulated irradiated cell of the invention, or a combination thereof), and a pharmaceutically acceptable carrier. In a specific embodiment, the term “pharmaceutically acceptable” means approved by a regulatory agency of the Federal or a state government or listed in the U.S. Pharmacopeia, or European Pharmacopeia, or other generally recognized pharmacopeia for use in animals, and more particularly in humans. The term “carrier” refers to a diluent, adjuvant, excipient, or vehicle with which the therapeutic agent is administered. The composition, if desired, can also contain minor amounts of pH buffering agents. Examples of suitable pharmaceutical carriers are described in “Remington's Pharmaceutical Sciences” by E.W. Martin. Such compositions will contain a prophylactically or therapeutically effective amount of a prophylactic or therapeutic agent preferably in purified form, together with a suitable amount of carrier so as to provide the form for proper administration to the subject. The formulation should suit the mode of administration. In a preferred embodiment, the pharmaceutical compositions are sterile and in suitable form for administration to a subject, preferably an animal subject, more preferably a mammalian subject, and most preferably a human subject.

The pharmaceutical composition of the invention may be in a variety of forms. These include, for example, solid, semi-solid, and liquid dosage forms, such as lyophilized preparations, liquids solutions or suspensions, injectable and infusible solutions, etc. The preferred form depends on the intended mode of administration and therapeutic application.

The administration of the cell population of the invention, or the pharmaceutical composition comprising same, to the subject in need thereof can be carried out by conventional means. In a particular embodiment, said cell population is administered to the subject by a method which involves transferring the cells to the desired tissue, either in vitro (e.g., as a graft prior to implantation or engrafting) or in vivo, to the animal tissue directly. The cells can be transferred to the desired tissue by any appropriate method, which generally will vary according to the tissue type. For example, cells can be transferred to graft by bathing the graft (or infusing it) with culture medium containing the cells. Alternatively, the cells can be seeded onto the desired site within the tissue to establish a population. Cells can be transferred to sites in vivo using devices such as catheters, trocars, cannulae, stents (which can be seeded with the cells), etc.

The cells of the invention can be irradiated before administration to the subject. This treatment makes cells incapable to proliferate or survive for long time periods in the subject. Thus, in a particular embodiment, the pharmaceutical composition of the invention comprises irradiated cells of the invention.

Also, the cells of the invention can be pre-stimulated with IFN-γ, prior to administration to the subject in order to reduce the time period between cell administration and IDO expression in the subject. Thus, in a particular embodiment, the pharmaceutical composition of the invention comprises IFN-γ-pre-stimulated cells of the invention.

Further, the cells of the invention can be both irradiated and pre-stimulated with IFN-γ, in any order, prior to administration to the subject. Thus, in a particular embodiment, the pharmaceutical composition of the invention comprises irradiated IFN-γ-pre-stimulated cells of the invention or IFN-γ-pre-stimulated irradiated cells of the invention.

The cell populations and pharmaceutical compositions of the invention can be used in a combination therapy. In a specific embodiment, the combination therapy is administered to a subject with an inflammatory disorder that is refractory to one or more anti-inflammatory agents. In another embodiment, the combination therapy is used in conjunction with other types of anti-inflammatory agents including, but not limited to, non-steroidal anti-inflammatory drugs (NSAIDs), steroidal anti-inflammatory drugs, beta-agonists, anticholingeric agents, and methyl xanthines. Examples of NSAIDs include, but are not limited to, ibuprofen, celecoxib, diclofenac, etodolac, fenoprofen, indomethacin, ketoralac, oxaprozin, nabumentone, sulindac, tolmentin, rofecoxib, naproxen, ketoprofen, nabumetone, etc. Such NSAIDs function by inhibiting a cyclooxygenase enzyme (e.g., COX-1 and/or COX-2). Examples of steroidal anti-inflammatory drugs include, but are not limited to, glucocorticoids, dexamethasone, cortisone, hydrocortisone, prednisone, prednisolone, triamcinolone, azulfidine, and eicosanoids such as thromboxanes, and leukotrienes. Monoclonal antibodies, such as Infliximab, can also be used.

In accordance with the above embodiment, the combination therapies of the invention can be used prior to, concurrently or subsequent to the administration of such anti-inflammatory agents. Further, such anti-inflammatory agents do not encompass agents characterized herein as lymphoid tissue inducers and/or immunomodulatory agents.

### Uses of the Cells of the Invention

An alternative aspect of the present invention provides the use of the cells of the invention or regulatory T-cells of the invention in the manufacture of a medicament for treating or repairing damaged tissue (preferably mesenchymal tissue), and/or for the treatment, modulation, prophylaxis, and/or amelioration of one or more symptoms associated with inflammatory and/or immune disorders, by administration of the cells or regulatory T-cells of the invention.

In a further aspect the present invention provides a pharmaceutical composition comprising of the cells or regulatory T-cells of the invention. Said pharmaceutical compositions are of use in the treatment, repair, prophylaxis, and/or amelioration of damaged tissues, or one or more symptoms associated with inflammatory and/or immune disorders such as but not limited to autoimmune diseases, inflammatory disorders, and immunologically mediated diseases including rejection of transplanted organs and tissues. In one embodiment of the invention the pharmaceutical composition may further comprise an antigen, group of antigens or cell types expressing and/or presenting said antigen or antigens. In one embodiment the antigen is selected from a group comprising of: a mixture of autoantigens derived from a patient suffering with autoimmunity, a peptide antigen, a nucleic acid, an altered peptide ligand, a recombinant protein or fragments thereof. In one embodiment said antigens are associated with arthritis, such as but not limited to collagen antigens. In an alternative embodiment said antigens are associated with Celiac Disease. Antigens associated with celiac disease are members of the gluten family including some forms of prolamins (such as but not limited to antigens of gliadins, hordeins, and/or secalins). Gluten and its components, glutanin and gliadin, are preferred antigens associated with Celiac disease. In a further embodiment said antigens are associated with multiple sclerosis, such as but not limited to myelin antigens and myelin component antigens such as myelin basic protein (MBP), myelin oligodendrocyte glycoprotein (MOG), proteolipid protein (PLP) and myelin glycolipids e.g. galactocerebroside. Methods for the isolation, purification and preparation of such antigens are known to the person skilled in the art.

The pharmaceutical composition of the invention comprises a prophylactically or therapeutically effective amount of the cells or regulatory T-cells of the invention, optionally antigen, and a pharmaceutical carrier. Examples of dosages and dosage regimes for each of these cell types are given above. Suitable pharmaceutical carriers are known in the art and are preferably those approved by a regulatory agency of the US Federal or a state government or listed in the U S Pharmacopeia, or European Pharmacopeia, or other generally recognized pharmacopeia for use in animals, and more particularly in humans. The term “carrier” refers to a diluent, adjuvant, excipient, or vehicle with which the therapeutic agent is administered. The composition, if desired, can also contain minor amounts of pH buffering agents. Examples of suitable pharmaceutical carriers are described in “Remington's Pharmaceutical Sciences” by E W Martin. Such compositions will contain a prophylactically or therapeutically effective amount of a prophylactic or therapeutic agent preferably in purified form, together with a suitable amount of carrier so as to provide the form for proper administration to the subject. The formulation should suit the mode of administration. In a preferred embodiment, the pharmaceutical compositions are sterile and in suitable form for administration to a subject, preferably an animal subject, more preferably a mammalian subject, and most preferably a human subject.

The pharmaceutical composition of the invention may be in a variety of forms. These include, for example, semi-solid, and liquid dosage forms, such as lyophilized preparations, liquid solutions or suspensions, injectable and infusible solutions, etc. As noted above, the pharmaceutical composition is preferably injectable.

It is preferred that the methods, medicaments, compositions, cells and regulatory T-cells of the invention are used for treating or repairing damaged tissue (preferably mesenchymal tissue), and/or for the treatment, modulation, prophylaxis, and/or amelioration of one or more symptoms associated with inflammatory and/or immune disorders. Accordingly the methods and cells of the invention are of use in the treatment of any disorder characterized by either or all of said symptoms. A representative non-exhaustive list of such disorders is provided in the definitions section. Particularly preferred is the use of the methods, medicaments, compositions and cells of the invention in the treatment of immune-mediated inflammatory diseases. Further preferred is the use of the methods, medicaments, compositions and cells of the invention in the treatment of diabetes mellitus, rheumatoid arthritis (RA), inflammatory bowel disease (IBD, including Crohn's disease and/or Ulcerative Colitis) and multiple sclerosis (MS). Even more particularly preferred is the use of the methods, medicaments, compositions and cells of the invention in the treatment of rheumatoid arthritis.

Wherein the method or composition of the invention comprises one or more antigens it is preferred that the method or composition is used in the treatment of the disorder associated with or induced by said antigen, for example wherein the antigen is collagen the method or composition may be used in the treatment of arthritis, wherein the antigen is a gluten component the methods or compositions may be used in the treatment of celiac disease, wherein the antigen is a myelin component the methods or compositions may be used to treat multiple sclerosis. Preferred compositions therefore comprise: MSC, preferably ASC, and collagen, for the treatment of arthritis; MSC, preferably ASC, and gluten and/or a gluten component, for the treatment of celiac disease; MSC, preferably ASC, and myelin and/or a myelin component, for the treatment of multiple sclerosis.

In a further aspect the present invention provides a kit comprising i) a medicament comprising the cells or regulatory T-cells of the invention and ii) instructions for the use thereof according to the methods of the present invention.

In a further embodiment said kit may further comprise of iii) one or more antigens.

### Method for Identifying Cells of the Invention

In a further embodiment the present invention provides a method for selection of cells of the invention comprising the following steps:
i) providing a population of mesenchymal stem cells comprising cells having the capacity to be differentiated into at least two cell lineages
ii) determining the presence or absence of markers CD112 and/or CD155 in cells or subpopulations of said population of adipose derived cells
iii) selecting the cells or subpopulations negative for the markers CD112 and/or CD155.

The population of mesenchymal stem cells provided in i) is most preferably a plastic adherent cell population isolated according to the methods as previously described. Most preferably said cell population comprises essentially of cells having the capacity to be differentiated into at least two cell lineages.

Briefly, the population of cells is obtained by conventional means from any suitable source of adipose tissue from any suitable animal, preferably humans, e.g., from human adipose tissue. The animal can be alive or dead, so long as connective tissue cells within the animal are viable. Typically, human adipose cells are obtained from living donors, using well-recognized protocols such as surgical or suction lipectomy. As liposuction procedures are so common, liposuction effluent is a particularly preferred source from which the cells of the invention can be derived. Thus, in a particular embodiment, the cells of the invention are from the stromal fraction of human adipose tissue obtained by liposuction.

The sample of adipose tissue is, preferably, washed before being processed to separate the cells of the invention from the remainder of the material. In a protocol, the sample of tissue is washed with physiologically-compatible saline solution (e.g., phosphate buffered saline (PBS)) and then vigorously agitated and left to settle, a step that removes loose matter (e.g., damaged tissue, blood, erythrocytes, etc) from the tissue. Thus, the washing and settling steps generally are repeated until the supernatant is relatively clear of debris. The remaining cells generally will be present in clumps of various sizes, and the protocol proceeds using steps gauged to degrade the gross structure while minimizing damage to the cells themselves. One method of achieving this end is to treat the washed lumps of cells with an enzyme that weakens or destroys bonds between cells (e.g., collagenase, dispase, trypsin, etc.). The amount and duration of such enzymatic treatment will vary, depending on the conditions employed, but the use of such enzymes is generally known in the art. Alternatively or in conjunction with such enzymatic treatment, the lumps of cells can be degraded using other treatments, such as mechanical agitation, sonic energy, thermal energy, etc. If degradation is accomplished by enzymatic methods, it is desirable to neutralize the enzyme following a suitable period, to minimize deleterious effects on the cells.

The degradation step typically produces a slurry or suspension of aggregated cells and a fluid fraction containing generally free stromal cells (e.g., red blood cells, smooth muscle cells, endothelial cells, fibroblast cells, and stem cells). The next stage in the separation process is to separate the aggregated cells from the cells of the invention. This can be accomplished by centrifugation, which forces the cells into a pellet covered by a supernatant. The supernatant then can be discarded and the pellet suspended in a physiologically-compatible fluid. Moreover, the suspended cells typically include erythrocytes, and in most protocols it is desirable to lyse them. Methods for selectively lysing erythrocytes are known in the art, and any suitable protocol can be employed (e.g., incubation in a hyper- or hypotonic medium, by lysis using ammonium chloride, etc.). Of course, if the erythrocytes are lysed, the remaining cells should then be separated from the lysate, for example by filtration, sedimentation, or density fractionation.

Regardless of whether the erythrocytes are lysed, the suspended cells can be washed, re-centrifuged, and resuspended one or more successive times to achieve greater purity. Alternatively, the cells can be separated on the basis of cell surface marker profile or on the basis of cell size and granularity.

Following the final isolation and resuspension, the cells can be cultured and, if desired, assayed for number and viability to assess the yield. Preferably, the cells will be cultured without differentiation, on a solid surface, using a suitable cell culture media, at the appropriate cell densities and culture conditions. Thus, in a particular embodiment, cells are cultured without differentiation on a solid surface, usually made of a plastic material, such as Petri dishes or cell culture flasks, in the presence of a suitable cell culture medium [e.g., DMEM, typically supplemented with 5-15% (e.g., 10%) of a suitable serum, such as fetal bovine serum or human serum], and incubated under conditions which allow cells to adhere to the solid surface and proliferate. After incubation, cells are washed in order to remove non-adhered cells and cell fragments. The cells are maintained in culture in the same medium and under the same conditions until they reach the adequate confluence, typically, about 80% cell confluence, with replacement of the cell culture medium when necessary. After reaching the desired cell confluence, the cells can be expanded by means of consecutive passages using a detachment agent such as trypsin and seeding onto a bigger cell culture surface at the appropriate cell density (usually 2,000-10,000 cells/cm²). Thus, cells are then passaged at least two times in such medium without differentiating, while still retaining their developmental phenotype, and more preferably, the cells can be passaged at least 10 times (e.g., at least 15 times or even at least 20 times) without losing developmental phenotype. Typically, the cells are plated at a desired density such as between about 100 cells/cm² to about 100,000 cells/cm² (such as about 500 cells/cm² to about 50,000 cells/cm², or, more particularly, between about 1,000 cells/cm² to about 20,000 cells/cm²). If plated at lower densities (e.g., about 300 cells/cm²), the cells can be more easily clonally isolated. For example, after a few days, cells plated at such densities will proliferate into an homogeneous population. In a particular embodiment, the cell density is between 2,000-10,000 cells/cm².

In step ii) of the method the presence or absence of markers CD112 and/or CD155 in cells or subpopulations of the population of adipose derived cells is determined. The clones or sub-populations, may be established using methods commonly used in the art for cloning cell populations. For example, a population of cells can be physically picked and seeded into a separate plate (or the well of a multi-well plate). Alternatively, the cells can be subcloned onto a multi-well plate at a statistical ratio for facilitating placing a single cell into each well (e.g., from about 0.1 to about 1 cell/well or even about 0.25 to about 0.5 cells/well, such as 0.5 cells/well). Of course, the cells can be cloned by plating them at low density (e.g., in a Petri dish or other suitable substrate) and isolating them from other cells using devices such as a cloning rings. The presence or absence of markers CD112 and/or CD155 may be determined by any means standard in the art such as but not limited to by means of flow cytometry.

In step iii) of the method the clones or subpopulations negative for the markers CD112 and/or CD155 are selected. As used herein, “negative” with respect to cell surface markers means that, in a cell population comprising the cells of the invention, less than 1%, preferably 0.9%, 0.8%, 0.7%, 0.6%, 0.5%, 0.4%, 0.3%, 0.2%, 0.1% or none of the cells show a signal for a specific cell surface marker in flow cytometry above the background signal, using conventional methods and apparatus (for example .a Beckman Coulter Epics XL FACS system used with commercially available antibodies and standard protocols known in the art). Commercially available and known monoclonal antibodies against said cell-surface markers (e.g., cellular receptors and transmembrane proteins) can be used to identify the cells of the invention.

### Uses of the Cells of the Invention

An alternative aspect of the present invention provides the use of the cells or regulatory T-cells of the invention in the manufacture of a medicament for treating or repairing damaged tissue (preferably mesenchymal tissue), and/or for the treatment, modulation, prophylaxis, and/or amelioration of one or more symptoms associated with inflammatory and/or immune disorders, by administration of the cells or regulatory T-cells of the invention.

In a further aspect the present invention provides a pharmaceutical composition comprising of the cells or regulatory T-cells of the invention. Said pharmaceutical compositions are of use in the treatment, repair, prophylaxis, and/or amelioration of damaged tissues, or one or more symptoms associated with inflammatory and/or immune disorders such as but not limited to autoimmune diseases, inflammatory disorders, and immunologically mediated diseases including rejection of transplanted organs and tissues. In one embodiment of the invention the pharmaceutical composition may further comprise an antigen, group of antigens or cell types expressing and/or presenting said antigen or antigens. In one embodiment the antigen is selected from a group comprising of: a mixture of autoantigens derived from a patient suffering with autoimmunity, a peptide antigen, a nucleic acid, an altered peptide ligand, a recombinant protein or fragments thereof. In one embodiment said antigens are associated with arthritis, such as but not limited to collagen antigens. In an alternative embodiment said antigens are associated with Celiac Disease. Antigens associated with celiac disease are members of the gluten family including some forms of prolamins (such as but not limited to antigens of gliadins, hordeins, and/or secalins). Gluten and its components, glutanin and gliadin, are preferred antigens associated with Celiac disease. In a further embodiment said antigens are associated with multiple sclerosis, such as but not limited to myelin antigens and myelin component antigens such as myelin basic protein (MBP), myelin oligodendrocyte glycoprotein (MOG), proteolipid protein (PLP) and myelin glycolipids e.g. galactocerebroside. Methods for the isolation, purification and preparation of such antigens are known to the person skilled in the art.

The pharmaceutical composition of the invention comprises a prophylactically or therapeutically effective amount of the cells or regulatory T-cells of the invention, optionally antigen, and a pharmaceutical carrier. Examples of dosages and dosage regimes for each of these cell types are given above. Suitable pharmaceutical carriers are known in the art and are preferably those approved by a regulatory agency of the US Federal or a state government or listed in the U S Pharmacopeia, or European Pharmacopeia, or other generally recognized pharmacopeia for use in animals, and more particularly in humans. The term “carrier” refers to a diluent, adjuvant, excipient, or vehicle with which the therapeutic agent is administered. The composition, if desired, can also contain minor amounts of pH buffering agents. Examples of suitable pharmaceutical carriers are described in “Remington's Pharmaceutical Sciences” by E W Martin. Such compositions will contain a prophylactically or therapeutically effective amount of a prophylactic or therapeutic agent preferably in purified form, together with a suitable amount of carrier so as to provide the form for proper administration to the subject. The formulation should suit the mode of administration. In a preferred embodiment, the pharmaceutical compositions are sterile and in suitable form for administration to a subject, preferably an animal subject, more preferably a mammalian subject, and most preferably a human subject.

The pharmaceutical composition of the invention may be in a variety of forms. These include, for example, semi-solid, and liquid dosage forms, such as lyophilized preparations, liquid solutions or suspensions, injectable and infusible solutions, etc. As noted above, the pharmaceutical composition is preferably injectable.

It is preferred that the methods, medicaments, compositions, cells and regulatory T-cells of the invention are used for treating or repairing damaged tissue (preferably mesenchymal tissue), and/or for the treatment, modulation, prophylaxis, and/or amelioration of one or more symptoms associated with inflammatory and/or immune disorders. Accordingly the methods and cells of the invention are of use in the treatment of any disorder characterized by either or all of said symptoms. A representative non-exhaustive list of such disorders is provided in the definitions section. Particularly preferred is the use of the methods, medicaments, compositions and cells of the invention in the treatment of immune-mediated inflammatory diseases. Further preferred is the use of the methods, medicaments, compositions and cells of the invention in the treatment of diabetes mellitus, rheumatoid arthritis (RA), inflammatory bowel disease (IBD, including Crohn's disease and/or Ulcerative Colitis) and multiple sclerosis (MS). Even more particularly preferred is the use of the methods, medicaments, compositions and cells of the invention in the treatment of rheumatoid arthritis.

Wherein the method or composition of the invention comprises one or more antigens it is preferred that the method or composition is used in the treatment of the disorder associated with or induced by said antigen, for example wherein the antigen is collagen the method or composition may be used in the treatment of arthritis, wherein the antigen is a gluten component the methods or compositions may be used in the treatment of celiac disease, wherein the antigen is a myelin component the methods or compositions may be used to treat multiple sclerosis. Preferred compositions therefore comprise: MSC, preferably ASC, and collagen, for the treatment of arthritis; MSC, preferably ASC, and gluten and/or a gluten component, for the treatment of celiac disease; MSC, preferably ASC, and myelin and/or a myelin component, for the treatment of multiple sclerosis.

### KITS

In another aspect, the invention refers to a kit comprising a cell population containing (i) cells of the invention and/or (ii) regulatory T-cells of the invention and/or (iii) irradiated cells of the invention and/or (iv) IFN-γ-pre-stimulated cells of the invention, and/or (v) irradiated IFN-γ-pre-stimulated cells of the invention, and/or (vi) IFN-γ-pre-stimulated irradiated cells of the invention. Kits of the invention may comprise one, two, three, four, five or all of such cell types.

In a further aspect the present invention provides a kit comprising i) a medicament comprising the cells or regulatory T-cells of the invention and ii) instructions for the use thereof according to the methods of the present invention.

In a further embodiment said kit may further comprise of iii) one or more antigens.

### EXAMPLES

The invention will now be described in more detail, by way of examples which in no way are meant to limit the scope of the invention, but, rather, these examples will serve to illustrate the invention with reference to the accompanying figures.

### Material and Methods

Culture of hASCs and hBM-MSCs

hASCs were isolated from lipoaspirates obtained from human adipose tissue from healthy adult donors were washed twice with PBS, and digested at 37° C. for 30 min with 18 U/ml of collagenase type I in PBS. The digested sample was washed with 10% of fetal bovine serum (FBS), treated with Amonium Chloride 160 mM, suspended in culture medium (DMEM containing 10% FBS), and filtered through a 40-μm nylon mesh. Cells were seeded onto tissue culture flasks and expanded at 37° C. and 5% CO2, changing the culture medium every 7 days. Cells were passed to a new culture flask when cultures reached 90% of confluence. In addition, hASCs were verified by staining with specific surface markers being positive for HLA-I, CD90, and CD105, and negative for HLA-11, CD40, CD80, CD86, and CD34. A pool from six healthy donors (three men and three women, aged between 35 and 47) was used in the study. Cells were used at passages 4-6. The hASCs were obtained after informed consent under the auspices of the appropriate Research and Ethics Committees.

Human bone marrow mesenchymal stromal cells (hBM-MSCs) were used as a MSC reference cell source. hBM-MSCs were purchased from Lonza, Inc. (Walkersville, Md., USA) and cultivated according to the supplier's recommendations in DMEM containing 10% FBS.

Phenotypic Analysis of hASCs by FACS

For flow cytometric analysis of hASC, cells were stained with the following mAbs: anti-HLA-ABC (G46-2.6), anti-HLA-DR (L243), anti-CD112 (R2.525), anti-CD155 (300907), anti-MICA/B (6D4) from BD Biosciences (San Jose, Calif., USA). The ULBPs were evaluated with anti-ULBP-1 (170818) anti-ULBP-2 (165903) and anti-ULBP-3 (166510) from R&D systems (Minneapolis, Minn., USA) by indirect immunofluorescence using an appropriate FITC-conjugated secondary antibody. The analysis of recombinant human protein chimeras (NKp30-, NKp44- and NKp46-IgG1 (Fc) from R&D Systems was performed as follows: 2×10⁵ hASCs were incubated with 100 μl of Fc Receptor Blocker (Innovex Biosciences, Richmond, Va., USA) for 10 minutes at room temperature. Following wash twice with wash buffer and resuspended in PBS containing 2% FCS. Cells were then incubated with 5 μg/ml of protein chimera or human IgG for 30 min at 4° C. Following three washes at 4° C. in PBS containing 2% FSC, cells were incubated for 30 min at 4° C. with a human IgG-Fc fragment-specific, FITC-conjugated goat antibody. After two washes, the cells were resuspended in PBS and analyzed by flow cytometry. The flow cytometric analysis was performed on a FACScan cytometer (BD Biosciences) after acquisition of 10⁵ events. Viable cells were selected using forward and side scatter characteristics and analyzed using CellQuest software (BD Biosciences). Isotype-matched negative control antibodies were used in all the experiments. The Mean Relative Fluorescence Intensity (MRFI) was calculated by dividing the mean fluorescent intensity (MFI) by its negative control.

### NK Cell Isolation and Culture

Peripheral blood mononuclear cells (PBMCs) from healthy donors were obtained by centrifugation over Histopaque-1077 (Sigma, St. Louis, Mo.) and washed with PBS. PBMCs were grown for five days in RPMI 1640 supplemented with 10% human serum, 1% glutamine, 1% penicillin, 1% non-essential amino acids, 1% Sodium Pyruvate from Cambrex Bio Science (Walkersville, Md. USA) and 500 U/ml of rhIL-2 obtained from the National Cancer Institute at Frederick. NK cells were purified by using a FACS Vantage cytometer (BD Biosciences) and the typical purity of the sorted populations was 95-98%. Purified NK cells were subsequently assayed for degranulation and IFN-γ production or tested in co-culture experiments.

### NK Cell Degranulation Assay

The surface expression of CD107a/b was analysed after 4 hours following activation of purified NK cells with target cells at ratio 1:1 in the presence of BD GolgiStop™ (BD Biosciences) and a mixture of FITC-labelled CD107a/b. NK cells were stained with PE labelled anti-CD56 (NCAM16.2) from BD Biosciences and analyzed by flow cytometry, measuring the frequency of CD107a/b expression.

### Interferon-γ Assay

IFN-γ assay was performed using purified NK cells co-cultured with target cells at 1:1 ratio in the presence of BD GolgiStop™ (BD Biosciences). After 8 hours of co-incubation, purified NK cells were stained with PE labelled anti-CD56 (NCAM16.2) from BD Biosciences, fixed and permeabilized using BD Cytofix/Cytoperm fixation/permeabilization kit (BD Biosciences). Finally, cells were stained with FITC-labelled anti-IFN-γ mAb (eBioscience, San Diego, Calif.) and flow cytometry analysis was performed by measuring the frequency of IFN-γ expression.

Co-Culture of NK Cells with hASCs and hBM-MSCs

To determine the inhibitory effect of hASCs and hBM-MSCs on NK cells, purified NK cells were co-cultured in the presence or absence of hASCs or hBM-MSCs by using direct co-culture or in a transwell plate system with a 0.4 μm pore size membrane (Corning Costar, Schiphol-Rijk, The Netherlands). After 72 hours with rhIL-2 at 100 U/ml, NK cells were harvested and subsequently tested in a degranulation assay against the NK cell-susceptible target cell line K562. The analysis by flow cytometry was performed by measuring the frequency of CD107a/b expression as described above. The supernatants from co-cultures were collected, spun down to remove cells or cell debris, frozen and stored at −20° C.

### Phenotypic Analysis of NK Cells

Purified NK cells were co-cultured at ratio 1:1 with hASCs or hBM-MSCs in direct contact or in a transwell co-culture system for 72 h. For flow cytometric analysis, NK cells were washed twice in PBS and stained with the appropriate combination of fluorescent-labeled mAbs. The following mAbs were used in this study: Peridinin chlorophyll protein (PerCP)-conjugated anti-CD3 (SK1); fluorescein isothiocyanate (FITC)-conjugated anti-CD56 (NCAM16.2) and phycoerythrin (PE)-conjugated anti-CD69 (HP-4B3), anti-NKp44 (p44-8.1), anti-NKp46 (9E2/NKp46), anti-CD226 (DX11), anti-NKG2D (1D11), anti-CD94 (HP-3D9), anti-CD69 (HP-4B3), anti-CD16 (NKP15) from BD Biosciences, anti-NKp30 (p30-15) from Miltenyi Biotec (Auburn, Calif., USA) and anti-CD244 (C1.7) from e-Bioscience (San Diego, Calif.).

For intracellular staining, after surface-marker labeling, cells were fixed and permeabilized using BD Cytofix/Cytoperm fixation/permeabilization kit (BD Biosciences). Cells were stained with anti-Perforin (SG9), anti-Granzyme A (CB9) and anti-Granzyme B (GB11) from BD Biosciences.

Flow cytometric analysis was performed on a FACScan cytometer (BD Bioscience) after acquisition of 10⁵-10⁶ events. Viable cells were selected using forward and side scatter characteristics and NK cells were gated on CD56+CD3− phenotype and analyzed using CellQuest software (BD Biosciences). Isotype-matched negative control antibodies were used in all the experiments. The Mean Relative Fluorescence Intensity (MRFI) was calculated by dividing the Mean Fluorescent Intensify (MFI) by its negative control.

### IDO Activity

IDO activity was measured by determining both Trp and Kyn concentrations in supernatants from co-cultures of NK cells with hASCs or hBM-MSCs. About 200 μL of supernatants was added to 50 μL of trichloroacetic acid 2M, vortexed, spun down (10 min at 13,000 rpm) and analyzed by HPLC (Waters 717plus Autosampler, Milford, Mass.).

### Statistical Analysis

The differences in the means of measures were compared by the Student's t test and paired test for 2-way ANOVA using SPSS software. A p value ≦0.05 was considered significant.

### Results

Expression of Ligands for NK Activating Receptors in hASCs and hBM-MSCs

A phenotypic analysis of ligands for NK activating receptors in hASCs, as compared to hBM-MSCs isolated from healthy donors was carried out.

The results show that hASCs expressed lower levels of HLA class-I molecule compared with hBM-MSCs and negative expression of HLA class-II. In both cases, they did not express CD48 molecule (ligand for CD244). The phenotypic analysis of ligands for NK activating receptors in hASCs showed a lower expression of CD112 and CD155 (ligands for DNAM-1) compared with hBM-MSCs.

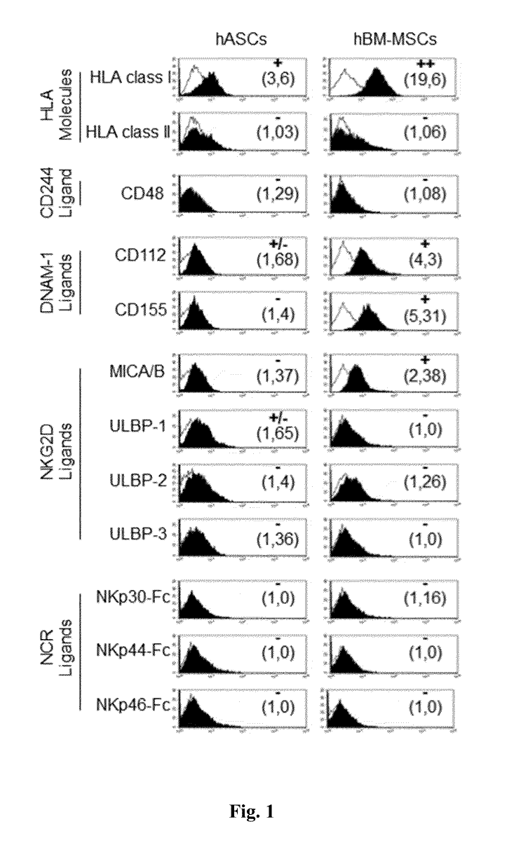

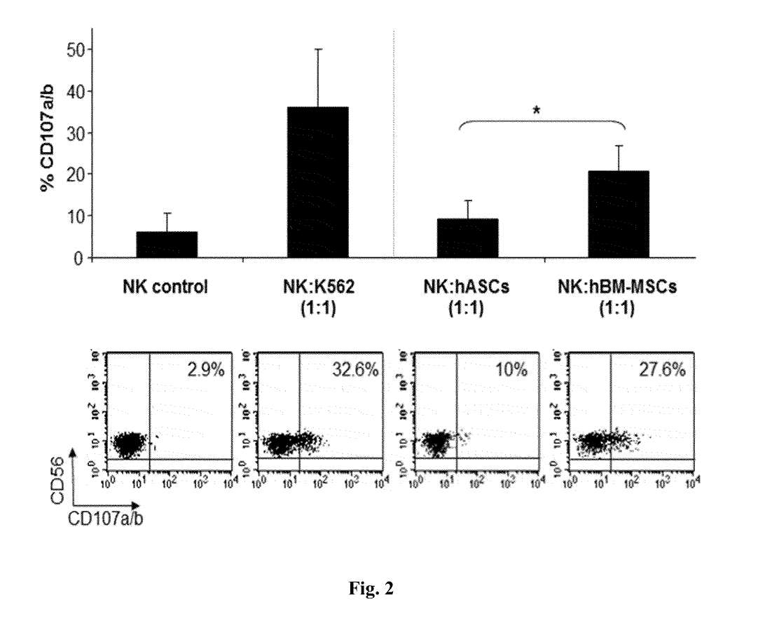

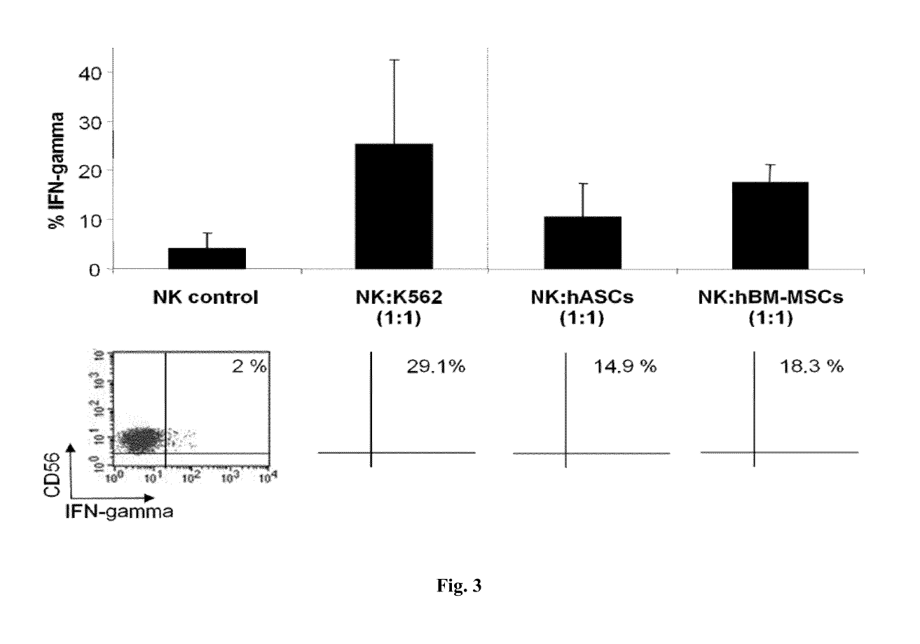

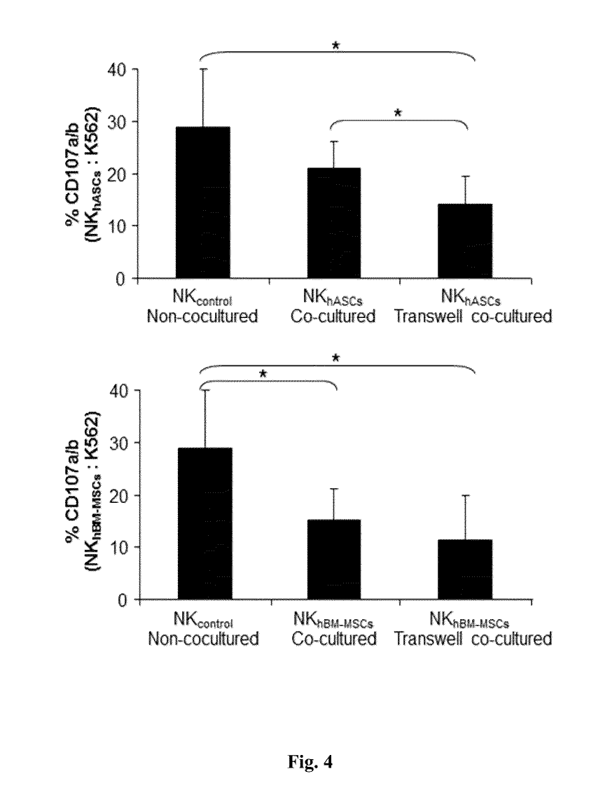

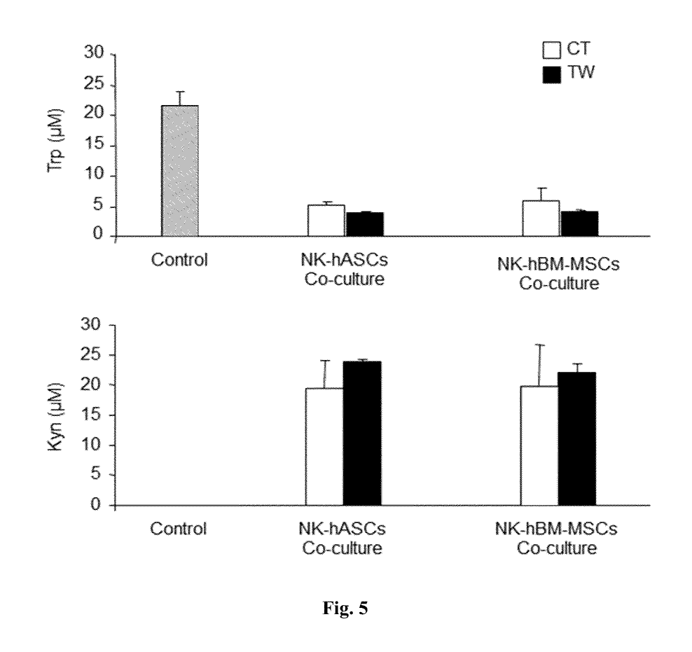

Furthermore, it was found that the expression of MICA/B and ULBPs (ligands for NKG2D) was very low or negative in hASCs and hBM-MSCs (FIG. 1). For the identification of ligands to natural cytotoxicity receptors (NCRs) by flow cytometry, chimeric NCRs were used to identify their surface expression in hASCs and hBMMSCs. Our results showed a negative expression of these ligands in both cell types (FIG. 1). The hASCs were negative for CD45, CD14, CD31, CD34, FCR1α, and 1B10 demonstrating the absence of potential contaminant cells that may potentially be found in the stromal vascular fraction. The hASCs were positive for CD29, CD59, CD73, CD90, and CD105 with no significant differences in the phenotype of pooled hASCs samples from different donors and individual samples (data not shown) and no significant differences after different passages.

Finally, to determine the expression levels of ligands for NK activating receptors in an inflammatory setting, hASCs and hBM-MSCs were stimulated with IFN-γ for 72 h (1, 10, and 100 U/mL). Three independent experiments showed that the expression level was not significantly increased in CD112, CD155, MICA/B, CD48, or ULBPs.

As expected, the expression of HLA class I and class II molecules was enhanced when these cells were treated with IFN-γ. After 72 h at 100 U/mL, the HLA class I expression (MRFI) increased in hASCs until 38.47-20.71 and 48.74-38.5 in hBM-MSCs. Moreover, the HLA class II in hASCs and hBM-MSCs switched to positive value reaching a MRFI of 2.44-1.21 and 2.21-1.32, respectively.

Low Degranulation Activity of NK Cells in Response to hASCs.

In order to compare the susceptibility of hASCs and hBMMSCs to NK cell-mediated lysis, degranulation assays were performed by the detection of CD107a/b surface molecule as previously described. The degranulation assay is a highly sensitive method, and its results are strictly correlated with NK cell cytotoxicity.

For these experiments, allogeneic NK cells were previously stimulated with rhIL-2 to increase their cytotoxic potential and sorted on the basis of CD56+CD3-phenotype. Both hASCs and hBM-MSCs were used as target cells in a degranulation assay. As negative and positive controls, NK cells alone and NK cells against the NK-susceptible target cell line K562 were used.

Although other ratios were used, to achieve maximal degranulation activity, the optimal effector:target (E:T) cell ratio in the degranulation assays was 1:1 (data not shown). The results demonstrated that degranulation rates in response to hASCs were very low and not statistically significant compared with degranulation in unstimulated NK cells (NK control). In contrast, hBM-MSCs induced a significantly enhanced NK cell degranulation response when compared with hASCs (FIG. 2).

To exclude the role of HLA class I-specific inhibitory receptors, HLA class I blocking was performed by pretreating the target cells with the HLA-class I specific monoclonal antibody W6/32 before co-culture. The results showed that antibody mediated masking of HLA class I did not increase NK cell degranulation against hASCs (data not shown).

hASCs and hBM-MSCs Induce Interferon-Gamma Production by NK Cells

In addition to degranulation assays, we examined the NK cell cytokine response to hASCs or hBM-MSCs. Similar to degranulation assays, IFN-γ production was analyzed in purified NK cells against hASCs, hBM-MSCs, and K562 (positive control). As negative controls, purified NK cells were cultured in the absence of target cells. The intracellular staining technique was used to assess IFN-γ production by NK cells. FIG. 3 shows that IFN-γ production was observed when hASCs or hBM-MSCs were used as target cells. Moreover, it is interesting to note that IFN-γ response was not statistically different when comparing hBM-MSCs with hASCs. A representative dot plot with the expression of IFN-γ over the NK cell population is also depicted in FIG. 3. The cytokine IFN-γ was also detected in supernatants from purified NK cells co-cultured with hASCs at a 1:1 ratio for 72 h by using the FlowCytomix human Th1/Th2 11plex kit (Bender MedSystem). The IFN-γ was secreted in NK/hASCs co-cultures (40-32.5 pg/mL) and not secreted by unstimulated NK cells or unstimulated hASCs (data not shown).

hASCs and hBM-MSCs Impair NK Cell Function Over Other Target Cells

To further study the possible modulatory effect of hASCs and hBM-MSCs on NK cells, co-culture experiments were carried out in direct contact co-cultures or by using transwell inserts. These experiments were designed to quantify the potential contribution of soluble factors and cell-to-cell contact in the modulation of NK activity.

Purified NK cells were co-cultured with hASC or hBMMSCs at a ratio 1:1 for 72 h and subsequently harvested and tested for degranulation capacity against a susceptible target cell line. Our results first revealed that the preincubation of NK cells with hASCs and hBM-MSCs were able to significantly decrease the NK degranulation capacity. Second, our results showed that precultured NK cells in transwell condition with hASCs had a significantly stronger inhibitory effect than cell-to-cell contact. Finally, with regard to the effect of hBM-MSCs over NK cells, comparable results to hASCs were obtained. Interestingly, no significant differences were observed between contact and transwell conditions in NK cells preincubated with hBM-MSCs (FIG. 4).

hASCs and hBM-MSCs Induce Phenotypic Changes in NK Cell

Subsequent to demonstrating the low capacity of hASCs cells to induce NK cell degranulation, the inventors analyzed the modifications over the NK cell phenotypic profile and determined whether cell-to-cell contact or only soluble factors were involved. For this purpose, activating receptors, NK cell markers, and effector molecules on NK cells co-cultured in the presence of hASC or BM-MSCs either in contact or in transwell conditions were analyzed.

Three independent experiments were performed, and the statistical analysis revealed that NK cells co-cultured in contact with hBM-MSCs showed a significantly reduced expression of DNAM-1, and very similar results (although not statistically significant) were obtained in NK cells cocultured in contact with hASCs (Table 1).

The activating receptor NKG2D was increased in NK cells co-cultured in Transwell with hASC and hBM-MSCs (this change was statistically significant only in the case of hASCs). Moreover, in direct-contact co-cultures, no differences were observed (Table 1). In the analysis of NCR repertoire, some changes were found (not statistically significant). A reduced NKp30 surface expression was observed when NK cells were cocultured in contact with hASCs and hBM-MSCs. In contrast, NKp46 expression was slightly increased (Table 1). Data revealed that NK cells in contact with hASCs and hBM-MSCs had a significant decrease of CD16; however, CD69 and CD94 expression was maintained in all the experimental conditions. Finally, intracellular molecules perforin, granzyme A, and granzyme B were also studied, and no changes were found for perforin and granzyme B; however, granzyme A was significantly reduced in NK cells co-cultured with hASCs both in contact and in transwells (Table 1).

TABLE 1
Phenotypic analysis of NK pre-sensitized with hASCs or hBM-MSCs in contact and in Transwell system

| Molecules | | NKhASCs | NKhASCs | NKhBM-MSCs | NKhBM-MSCs |
|---|---|---|---|---|---|
| (MRFI) | NK control | Co-culture CT | Co-culture TW | Co-culture CT | Co-culture TW |
| CD244 | 2.73 ± 0.82 | 2.64 ± 0.93 | 2.18 ± 0.63 | 2.21 ± 0.32 | 1.98 ± 0.30 |
| DNAM-1 | 7.59 ± 1.70 | 4.05 ± 1.82 | 7.52 ± 1.31 | 3.89 ± 1.36 | 6.60 ± 2.42 |
| NKG2D | 12.53 ± 5.16 | 14.52 ± 3.53 | 17.21 ± 6.94 | 11.24 ± 2.88 | 16.98 ± 9.71 |
| NKp30 | 16.63 ± 13.68 | 9.28 ± 7.02 | 10.41 ± 6.15 | 8.50 ± 6.46 | 12.41 ± 10.84 |
| NKp44 | 1.21 ± 1.00 | 1.59 ± 0.64 | 1.76 ± 0.83 | 1.54 ± 0.82 | 1.84 ± 1.12 |
| NKp46 | 6.89 ± 4.16 | 9.63 ± 7.09 | 9.79 ± 6.43 | 10.00 ± 7.29 | 8.25 ± 5.77 |
| CD16 | 81.99 ± 43.81 | 59.76 ± 13.05 | 80.80 ± 50.25 | 55.77 ± 7.31 | 74.92 ± 41.24 |
| CD69 | 5.82 ± 1.89 | 7.26 ± 2.08 | 6.10 ± 1.72 | 6.72 ± 3.33 | 6.10 ± 2.15 |
| CD94 | 104.03 ± 55.02 | 103.52 ± 35.51 | 102.84 ± 50.48 | 105.32 ± 32.61 | 97.32 ± 52.01 |
| Perforin | 31.98 ± 9.74 | 29.96 ± 8.51 | 30.65 ± 8.87 | 32.43 ± 10.31 | 31.16 ± 8.60 |
| Granzyme A | 10.47 ± 2.96 | 7.07 ± 2.83 | 8.49 ± 3.29 | 8.90 ± 4.81 | 9.45 ± 3.97 |
| Granzyme B | 4.77 ± 0.70 | 5.19 ± 0.92 | 4.67 ± 0.35 | 5.79 ± 1.31 | 4.50 ± 0.64 |

IDO is Induced by hASCs and hBM-MSCs in Response to NK Cells

Expression of IDO, a Trp catabolizing enzyme, contributes to the immunoregulatory functions of MSCs and is known to be involved in immunosuppression of effector cells. IDO expression is induced after IFN-γ stimulation. As previously shown in FIG. 3, hASCs and hBM-MSCs induced IFN-γ production by NK cells. We hypothesized that soluble factors released by NK cells could trigger IDO expression in hASCs and hBM-MSCs, which could play a role in the modulation of NK activity. Therefore, we measured by HPLC, IDO activity after co-culture.

As shown in FIG. 5, NK cells did not show IDO activity (measured as degradation of Trp and accumulation of its catabolic product Kyn, However, we found that concentrations of Trp gradually decreased, with the concomitant accumulation of Kyn when NK cells were co-cultured with hASCs or hBM-MSCs in contact or transwell conditions.

### CONCLUSION

The in vitro results demonstrate that hASCs display optimal characteristics for adoptive cell therapy in an allogeneic setting. First, the most important consequence of the lower expression of ligands for NK activating receptors would be their increased resistance to NK-mediated recognition. In comparison to hBM-MSCs, hASCs were more protected from allogeneic NK lysis. This would allow them to remain in the host for an extended period of time. Second, we suggest that mechanisms of hASCs for inducing tolerance in NK cells can be mediated by soluble factors. The IFN-γ secreted by NK cells during NK/MSCs crosstalk may induce IDO expression and other factors such as PGE2 that may exert a synergistic effect in the immunosuppressive activity.

In summary, this study provides a biological and therapeutic significance for increasing our understanding on the interactions between NK cells and adoptively transferred hASCs.

1.-11. (canceled)

12. An isolated mesenchymal stem cell population characterised in that the cells of said population do not express CD112 and/or CD155.

13. The cell population according to claim 12, characterised in that it is positive for at least one and preferably all the following cell surface markers: CD11b, CD11c, CD14, CD45, HLAII, CD31, CD34, CD45, 1B10 (αFSP), FceR1α and CD133.

14. A method for isolating a cell population according to claim 12 comprising the steps of:
(i) preparing a cell suspension from a sample of adipose tissue;
(ii) recovering the cells from said cell suspension;
(iii) incubating said cells in a suitable cell culture medium on a solid surface under conditions which allow cells to adhere to the solid surface and proliferate;
(iv) removing non-adhered cells;
(v) selecting the cells which after being passaged at least twice in such medium remain adhered to said solid surface;
(vi) determining the presence or absence of determining the presence or absence of markers CD112 and/or CD155 in individual cells or subpopulations; and
(vii) selecting the cells or subpopulations negative for the markers CD112 and/or CD155

15. A method for the preparation of a T-reg cell population which comprises:
(a) contacting a cell population according to claim 12 with peripheral blood leukocytes, and
(b) selecting the T-reg cell population.

16. An isolated T-reg cell population obtainable according to the method of claim 15.

17. A pharmaceutical composition comprising a cell population according to claim 12 and a pharmaceutically acceptable carrier.

18. A method for preventing, treating, or ameliorating one or more symptoms associated with autoimmune diseases, inflammatory disorders, or immunologically mediated diseases, in a subject suffering from said disorders or diseases, which comprises administering to said subject in need of such treatment of a prophylactically or therapeutically effective amount of a cell population according to claim 12.

19. The method according to claim 18, wherein said inflammatory disease is a chronic inflammatory disease.

20. Method according to claim 19, wherein said chronic inflammatory disease is selected from the group consisting of Inflammatory Bowel Disease (IBD) and Rheumatoid Arthritis (RA).

21. A method for selection of cells according to claim 12 comprising the following steps:
i) providing a population of mesenchymal stem cells comprising cells having the capacity to be differentiated into at least two cell lineages;
ii) determining the presence or absence of markers CD112 and/or CD155 in cells or subpopulations of said population of adipose derived cells; and
iii) selecting the cells or subpopulations negative for the markers CD112 and/or CD155.

22. A method for preventing, treating, or ameliorating one or more symptoms associated with autoimmune diseases, inflammatory disorders, or immunologically mediated diseases, in a subject suffering from said disorders or diseases, which comprises administering to said subject in need of such treatment of a prophylactically or therapeutically effective amount of a T-reg cell population according to claim 16.

23. The method according to claim 22, wherein said inflammatory disease is a chronic inflammatory disease.

24. The method according to claim 23, wherein said chronic inflammatory disease is selected from the group consisting of Inflammatory Bowel Disease (IBD) and Rheumatoid Arthritis (RA).

25. A method for preventing, treating, or ameliorating one or more symptoms associated with autoimmune diseases, inflammatory disorders, or immunologically mediated diseases, in a subject suffering from said disorders or diseases, which comprises administering to said subject in need of such treatment of a prophylactically or therapeutically effective amount of a pharmaceutical cell composition according to claim 17.

26. The method according to claim 25, wherein said inflammatory disease is a chronic inflammatory disease.

27. Method according to claim 26, wherein said chronic inflammatory disease is selected from the group consisting of Inflammatory Bowel Disease (IBD) and Rheumatoid Arthritis (RA).


SEQUENCE LISTING (2 sequences)

SEQ ID NO: 1 - protein, 551 aa, Homo sapiens
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 2 - protein, 417 aa, Homo sapiens
MARAMAAAWPLLLVALLVLSWPPPGTGDVVVQAPTQVPGFLGDSVTLPCYLQVPNMEVTHVSQLTWARHGESGSMAVFHQTQGPSYSESKRLEFVAARLGAELRNASLRMFGLRVEDEGNYTCLFVTFPQGSRSVDIWLRVLAKPQNTAEVQKVQLTGEPVPMARCVSTGGRPPAQITWHSDLGGMPNTSQVPGFLSGTVTVTSLWILVPSSQVDGKNVTCKVEHESFEKPQLLTVNLTVYYPPEVSISGYDNNWYLGQNEATLTCDARSNPEPTGYNWSTTMGPLPPFAVAQGAQLLIRPVDKPINTTLICNVTNALGARQAELTVQVKEGPPSEHSGISRNAIIFLVLGILVFLILLGIGIYFYWSKCSREVLWHCHLCPSSTEHASASANGHVSYSAVSRENSSSQDPQTEGTR